(19) United States
(12) Patent Application Publication
(10) Pub. No.: US 2021/0252186 A1
(43) Pub. Date: Aug. 19, 2021

(54) AN ADHESIVE LAYERED MATRIX AND USES THEREOF

(71) Applicant: NURAMI MEDICAL LTD., Nazareth (IL)

(72) Inventors: Amir BAHAR, Kiryat Tivon (IL); Nora NSEIR MANASSA, Haifa (IL); Hanna DODIUK KENIG, Haifa (IL)

(21) Appl. No.: 17/056,130

(22) PCT Filed: May 27, 2019
(86) PCT No.: PCT/IL2019/050604

§ 371 Date: Nov. 17, 2020

Related U.S. Application Data
(60) Provisional application No. 62/677,006, filed on May 27, 2018.

(51) Int. Cl. A61L 24/04; A61L 24/00

(52) U.S. Cl. CPC A61L 24/043; A61L 24/0036

(57) ABSTRACT

Compositions-of-matter comprising a matrix made of at least one elastic layer and at least one viscoelastic layer and an adhesive promoter and/or adhesive layer, are disclosed. The compositions-of-matter are characterized by high water-impermeability and optionally by self-recovery. Processes of preparing the compositions-of-matter and uses thereof as tissue substitutes or for repairing damaged tissues are also disclosed.

### CROSS REFERENCE TO RELATED APPLICATIONS

This application claims the benefit of priority from U.S. Provisional Patent Application No. 62/677,006 filed on May 27, 2018 entitled “AN ADHESIVE LAYERED MATRIX AND USES THEREOF”, the contents of which are incorporated herein by reference in their entirety.

### FIELD OF THE INVENTION

The present invention, in some embodiments thereof, relates to an elastic and adhesive layered matrix and to uses thereof as a tissue substitute.

### BACKGROUND OF THE INVENTION

Leakage of liquid or air from or into damaged tissue is a potentially life-threatening condition which may occur as a result of a wide variety of circumstances, including surgery and traumatic injury.

Soft tissues are particularly prone to damage. Additionally, these tissues sometimes create various compartments that hold liquid or air (e.g., lungs, blood vessels, dura matter, urinary bladder, etc.), and when damaged, their impairment can extend to other areas as well. Furthermore, because of the mechanical nature of these tissues, the attachment of a matrix with sutures or staples can cause damage in and of itself, e.g., to prevent proper sealing, to increase the probability of bacterial infection or to reduce the rate of recovery or recuperation. Examples of such soft tissues include dura mater, brain tissue, retina, skin tissue, hepatic tissue, pancreatic tissue, connective tissue, muscle tissue, cardiac tissue, vascular tissue, renal or urogenital tissue, pulmonary tissue, gonadal tissue, hematopoietic tissue, digestive tract tissue (such as colon or stomach) and fat tissue.

When these tissues are damaged, use of a matrix may be desired so as to: initially seal the damaged area; prevent leakage from the damaged tissue; reduce the probability of bacterial penetration; provide a scaffold for rapid and ordered generation of a new tissue; provide a safe structural and mechanical support, and all of the above without the need to use staples or sutures. Using the latter could cause further damage and might slow the time it takes to attach the matrix since the tissue or organ is moving and generating mechanical forces such as pulling, pushing and extension forces.

Adhesion to tissue without suturing may be provided by an adhesive (e.g., applied on a surface) which promotes cell growth attachment (e.g., growth factors, extracellular matrix proteins, and/or other proteins).

Polymerizable compositions have various used in polymer adhesives, for example as dental materials or as adhesives for holding reconstructive elements in place.

WO 2015/092797 discloses compositions-of-matter comprising a matrix made of one or more elastic layers and one or more viscoelastic layers, the compositions-of-matter being characterized by high water-impermeability and optionally by self-recovery.

### SUMMARY OF THE INVENTION

According to an aspect of some embodiments of the present invention there is provided a composition-of-matter comprising a multi-layer matrix, the matrix comprising (i) at least one layer of an elastic polymeric material (ii) at least one layer of a viscoelastic polymeric material, and (iii) at least one adhesive promoter and/or adhesive layer, applied on, or within, at least one surface of at least one of the elastic polymeric material.

In some embodiments, the matrix comprises two layers of an elastic polymeric material, wherein the layer of viscoelastic polymeric material is interposed between the two layers of an elastic polymeric material.

In some embodiments, the adhesive layer comprises one or more materials selected from: thrombin, fibrinogen, an albumin, gelatin coating, a polysaccharide or a derivative thereof, poly(vinyl acetate), poly(vinyl pyrrolidone, a polyester, polyethyleneglycol, polyaminoacid, poly(glutamic acid), poly (L-aspartic acid), or any combination thereof.

In some embodiments, the adhesive layer comprises one or more growth factors.

In some embodiments, the elastic polymeric material comprises a polymer characterized by a glass transition temperature and/or melting point at a temperature above 40° C. In some embodiments, each layer of the elastic polymeric material is a porous layer characterized by a porosity of at least 50%. In some embodiments, the polymeric fibers are characterized by a mean diameter in a range of from 0.001 to 30 μm.

In some embodiments, the layer of the viscoelastic polymeric material comprises a polymer characterized by a glass transition temperature and/or melting point at a temperature below 40° C.

In some embodiments, the layer of a viscoelastic polymeric material is characterized by a loss tangent (G″/G′) at a temperature of 10° C. and frequency of 0.1 Hz which is in a range of from 0.01 to 4.

In some embodiments, the layer of a viscoelastic polymeric material is characterized by at least one of: a) a storage shear modulus (G′) in a range of from 0.01 to 10 MPa, at a temperature of 10° C. and frequency of 0.1 Hz; and b) a loss shear modulus (G″) in a range of from 0.0001 to 2 MPa, at a temperature of 10° C. and frequency of 0.1 Hz.

In some embodiments, the polymeric fibers comprise electrospun elastic polymeric material.

In some embodiments, the elastic polymeric material is biocompatible. In some embodiments, each of the layers of an elastic polymeric material is characterized by a thickness in a range of from 10 to 500 μm. In some embodiments, each of the layers of an elastic polymeric material is characterized by an elastic modulus in a range of from 1 kPa to 10 MPa. In some embodiments, each of the layers of an elastic polymeric material is characterized by an elongation at failure of at least 100%. In some embodiments, each of the layers of an elastic polymeric material is characterized by an ultimate tensile strength of at least 0.05 MPa. In some embodiments, each of the layers of an elastic polymeric material is characterized by a recovery of at least 75%.

In some embodiments, the viscoelastic polymeric material comprises poly(lactic acid-co-ε-caprolactone).

In some embodiments, the elastic polymeric material is selected from the group consisting of poly(lactic acid-co-ε-caprolactone), poly(ε-caprolactone-co-L-lactic acid-co-glycolic acid-co-trimethylene carbonate), mixtures of poly(lactic acid-co-ε-caprolactone) and poly(lactic acid), and mixtures of poly(ε-caprolactone-co-L-lactic acid-co-glycolic acid-co-trimethylene carbonate) and poly(lactic acid).

In some embodiments, the matrix is characterized by a water-permeability of less than 1 ml per hour per cm² upon exposure to an aqueous liquid at a pressure of 40 mmHg.

In some embodiments, the disclosed composition-of-matter comprises the adhesion promoter.

In some embodiments, the disclosed composition-of-matter comprises the adhesive layer.

In some embodiments, the adhesion promoter is incorporated in at least one portion of the at least one adhesive layer. In some embodiments, the adhesion promoter is deposited on at least one surface of the at least one adhesive layer.

In some embodiments, the adhesive layer is or comprises a polymerizable resin selected from the group consisting of a polymerizable monomer and a filler. In some embodiments, the at least one adhesion promoter comprises a vinyl group. In some embodiments, the at least one adhesion promoter is or comprises a silane compound or a derivative thereof. In some embodiments, the derivative of the silane comprises one or more functional groups selected from the group consisting of amine, epoxy, or polyolefin. In some embodiments, the derivative of the silane is selected from the group consisting of vinyltriethoxysilane, triethoxysilylpropyl, and 3-aminopropyltriethoxysilane.

In some embodiments, the polymerization of the polymerizable resin is affected by irradiation. In some embodiments, the irradiation is thermal irradiation. In some embodiments, the irradiation is light irradiation.

In some embodiments, the polymerizable monomer is selected from the group consisting of methyl methacrylate, triethylene glycol dimethacrylate, 2-hydroxyethyl methacrylate, hexanediol methacrylate, dodecanediol dimethacrylate, bisphenol-A-dimethacrylate, bisphenylglycidyl methacrylate, mono- and urethane acrylate, urethane dimethacrylate, epoxy-acrylate and urethano-acrylate.

According to an aspect of some embodiments of the present invention there is provided an article-of-manufacture comprising the disclosed composition-of-matter in any embodiment thereof. In some embodiments the disclosed article-of-manufacture is a medical device. In some embodiments the disclosed article-of-manufacture is a tissue substitute. In some embodiments the disclosed article-of-manufacture is identified for use in repairing tissue damage.

In some embodiments the disclosed article-of-manufacture is identified for use in a treatment selected from the group consisting of dural repair, hernia repair, internal and/or topical wound closure, skin closure and/or repair, sealing tissues and/or organs in order to contain bodily fluids or air, sealing an anastomosis, inhibition of post-surgical adhesions between tissues, promotion of hemostasis, and administration of a therapeutically effective agent. In some embodiments the disclosed article-of-manufacture is for use in repairing and/or substituting a biological tissue.

According to an aspect of some embodiments of the present invention there is provided a method of repairing and/or substituting a biological tissue in a subject in need thereof, the method comprising contacting the biological tissue with the disclosed article-of-manufacture in any embodiment thereof, and affixing at least a portion of article-of-manufacture in/on a biological tissue thereby repairing and/or substituting the biological tissue.

In some embodiments, the affixing is performed by curing, In some embodiments the curing is thermal curing. In some embodiments, the affixing is performed by light radiation the adhesion promoter.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings and images. With specific reference now to the drawings and images in detail, it is stressed that the particulars shown are by way of example and for purposes of illustrative discussion of embodiments of the invention. In this regard, the description taken with the drawings and images makes apparent to those skilled in the art how embodiments of the invention may be practiced.

In the drawings:

FIG. 1 presents a scheme of an examplary integral layered matrix that contains an elastic fibrous layer, a viscoelastic layer and an adhesive layer;

FIG. 2 presents a scheme of the integral layered matrix with an adhesive layer that can be activated with radiation curing in selected area;

FIG. 3 presents a photograph showing samples (treated and non-treated patches) after performing a lap shear test, as described in the Examples section hereinbelow;

FIG. 4 presents a photograph showing lap shear samples prepared using strips of nanofiber patches; and

FIG. 5 presents a photograph showing lap shear samples bonded with the adhesive showing a substantial elongation during the tests.

### DETAILED DESCRIPTION OF THE INVENTION

The present invention, in some embodiments thereof, relates to tissue substitutes, and more particularly, but not exclusively, to an elastic and adhesive layered matrix and to uses thereof as a tissue substitute. Further, the present inventors have uncovered matrices which, upon selective surface activation (e.g., by radiation, or thermal activation), can exhibit the ability to form robust adhesion with biological tissues as well as improved mechanical strength, flexibility and/or impermeability.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The disclosed matrices, can respond to punctures such as those formed by suturing or stapling in a manner which limits the detrimental effects thereof.

Embodiments of the present invention relate to liquid-impermeable layered matrices which exhibit a unique combination of mechanical and rheological properties and to uses thereof in a variety of medical applications, and specifically, but not exclusively, as implants, and particularly as tissue substitutes such as, but not limited to, dura substitutes. Embodiments of the present invention further relate to recoverable matrices, which upon being subjected to suturing or stapling, self-recover so as to seal the holes formed by such procedures.

As used herein, the term “composition-of-matter” includes a matrix which is also referred to herein interchangeably as a “core matrix”, and may optionally further include additional components, ingredients and/or layers as described herein, according to any of the respective embodiments.

As used herein, the term “multi-layer” refers to a presence of at least two distinct layers. The distinct layers may differ, for example, in chemical composition, molecular configuration (e.g., degree and type of crystallinity), physical structure and/or mechanical properties.

Herein, the term “matrix” (including “core matrix”), when used in the context of a composition-of-matter comprising a multi-layer matrix as described herein, refers to the one or more elastic layers and viscoelastic layers (as described herein, according to any of the respective embodiments) and further includes any materials incorporated within and/or interposed between the elastic and/or viscoelastic layers.

The layered matrices provided herein comprise two or more layers, each made of a polymeric material, wherein one or more of these layers exhibit high elasticity and additional, one or more layers exhibit high viscoelasticity, and further comprise one or more layers capable of forming an adhesion with a biological tissue or surface.

As exemplified herein, layered matrices such as described herein (also referred to herein as “patches”) can be formed from biodegradable and biocompatible materials, while exhibiting considerable mechanical strength, a high degree of elasticity and flexibility, ease of handling, an ability to be folded (as may be useful for laparoscopic surgical procedures) without permanent deformation (e.g., without creasing), low density (which may decrease inflammation and infection), and a high degree of water-impermeability suitable for creating a tight seal, preventing fluid leakage, and preventing bacterial and viral infections.

According to an aspect of some embodiments of the invention, there is provided a composition-of-matter comprising a multi-layer matrix, the matrix comprising at least one layer, (or in some embodiments at least two layers) of an elastic polymeric material and at least one layer of a viscoelastic polymeric material attached to the layer of an elastic polymeric material, or interposed between two of the layers of an elastic polymeric material. In some embodiments, the composition-of-matter further comprises at least one adhesive material (also referred to “adhesive”).

According to an aspect of some embodiments of the present invention there is provided a composition-of-matter comprising a multi-layer matrix, the matrix comprising one or more from (i) and (ii):
(i) at least one, or, in some embodiments, at least two, elastic polymeric materials;
(ii) at least one layer of a viscoelastic polymeric material;
   and at least one material from (a) and (b):
      (a) at least one adhesive promoter;
      (b) at least one adhesive layer or additive,
         wherein:

(1) the layer of the viscoelastic polymeric material is attached to a layer of the elastic polymeric material, or, in some embodiments, interposed between two of the layers of the elastic polymeric material;

(2) each of the layers of the elastic polymeric material is independently made of polymeric fibers, and wherein:

(3) the adhesion promotor or the at least one adhesive layer are applied on at least one surface of at least one of the elastic polymeric material.

In some embodiments, the adhesive layer is a substance applied onto a multi-layer matrix and optionally is a product of surface modification of the multi-layer matrix. In some embodiments the adhesive layer is embedded in/on the elastic polymeric material or layer.

In some embodiments, the adhesive layer is obtained by a surface modification technique such as, without limitation, surface coating, bulk blending, plasma surface treatment (optionally with oxygen plasma, ammonia plasma, argon plasma or air plasma), exposure to flames, mechanical treatment, corona discharge, wet-chemical treatment and/or surface grafting (e.g., of monomers or polymers, optionally poly(N-isopropylacrylamide), poly(acrylic acid) and/or poly(amino acids)).

In some embodiments, and without being bound by any particular mechanism, the surface modification (e.g., plasma surface treatment, surface grafting) increases the hydrophilicity of a surface of the patch by altering the electrostatic charge of the surface.

In some embodiments, the adhesive layers comprises an adhesive substance (e.g., synthetic or biological in origin) in dry form, which, in some embodiments, is applied by coating at least a portion of the surface of the multi-layer matrix with the adhesive.

In some embodiments, the adhesiveness of a substance (e.g., additive) is enhanced upon hydration, for example, upon contact with moist tissue.

In some embodiments, the adhesive additive is optionally a dry combination of: thrombin, fibrinogen (which may interact upon hydration to form fibrin), an albumin, gelatin coating (optionally formed by electrospinning), a polymer (optionally a polysaccharide or chemically-modified polysaccharide, poly(vinyl acetate), poly(vinyl pyrrolidone, a polyester, polyethyleneglycol, polyethylene glycol (PEG; e.g., multi-arm PEG), polyaminoacid, poly(glutamic acid), poly (L-aspartic acid), or any combination thereof.

In some embodiments, the adhesive additive may include a functional group, for example, and without limitation, an imido ester, p-nitrophenyl carbonate, N-hydroxysuccinimide (NHS) ester, epoxide, isocyanate, acrylate, vinyl sulfone, orthopyridyl-disulfide, maleimide, or activated ester (e.g., thio-ester, peu-oroalkyl ester, pentu-orophenol ester, acid chloride, or anhydride, o-pyridyl-di sulfide), a thiol, multi-arms polyethylene glycol (optionally, 4-arm, to 8-arm), aldehyde and/or iodoacetamide group) that may react with a surface protein to form bonding.

In some embodiments, the amount of adhesive is controlled such that the adhesion strength of the matrix (as evaluated by the adhesive strength of the multi-layer matrix adhered to a biological fascia undergoing a burst pressure test) is at least 10 mmHg.

In some embodiments, the adhesive is selected to facilitate cell attachment and/or proliferation.

In some embodiments, the adhesive comprises a coating of growth factors and/or a layer of biocompatible nanofibers, optionally formed by electrospinning.

In some embodiments, the nanofibers optionally comprises synthetic polymers and/or co-polymers (e.g. polyesters) and/or biological polymers (for example, and without limitation, gelatin, collagen, elastin, laminin and/or fibronectin).

In some embodiments, the adhesive has a thickness in a range of from 1 to 400, or from 1 to 200 μm, or in some embodiments, in a range of from 1 to 100 μm, or, in some embodiments, in a range of from 50 to 100 μm.

In some embodiments of any one of the embodiments described herein, the matrix is defined by elastic layers (as described herein, according to any of the respective embodiments) and includes any materials (including, but not limited to, a viscoelastic layer according to any of the respective embodiments described herein) incorporated within and/or interposed between the elastic layers.

As used herein, the phrase “elastic layer” refers to a layer of material, wherein the layer exhibits elasticity. Herein, the terms “elasticity” and “elastic” refer to a tendency of a material (optionally in a form of a layer) to return to its original shape after being deformed by stress, for example, a tensile stress and/or shear stress, at an indicated temperature or at a temperature of 37° C. (in contexts wherein no temperature is indicated).

As used herein, the phrase “viscoelastic layer” refers to a layer of material, wherein the layer exhibits viscoelasticity.

An elastic layer according to any one of the embodiments described in this section described in this section may be combined with a viscoelastic polymeric material and/or viscoelastic layer according to any one of the respective embodiments described herein.

In some embodiments of any one of the embodiments described herein, the elastic layer is a porous layer. Herein, the phrase “porous layer” refers to a layer which comprises voids (e.g., in addition to polymeric material described herein), for example, the space between the polymeric material is not filled in by an additional substance. However, porous layers may optionally comprise an additional substance in the spaces between the polymeric material, provided that at least a portion of the volume of the voids is not filled in by the additional substance.

Porous layers may be, for example, in a form of fibers (e.g., woven or non-woven fibers, a foam and/or a sponge. Many suitable techniques will be known to the skilled practitioner for preparing a polymeric material in porous form, including, without limitation, various techniques for spinning fibers, use of a gas to form a foam, and drying (e.g., lyophilizing) a suspension of polymeric material.

In some embodiments of any one of the embodiments described herein relating to one or more porous layers (e.g., porous elastic layers), the porous layers are characterized by a porosity of at least 50% (e.g., from 50 to 99%). In some such embodiments, the porous layers are characterized by a porosity of at least 60% (e.g., from 60 to 99%). In some such embodiments, the porous layers are characterized by a porosity of at least 70% (e.g., from 70 to 99%). In some such embodiments, the porous layers are characterized by a porosity of at least 80% (e.g., from 80 to 99%). In some such embodiments, the porous layers are characterized by a porosity of at least 90% (e.g., from 90 to 99%). In some such embodiments, the porous layers are characterized by a porosity of about 90%.

As shown in the Examples section herein, the present inventors have surprisingly uncovered that even a highly porous elastic layer reduces matrix water-permeability considerably.

Herein, the term “porosity” refers to a percentage of the volume of a substance (e.g., an elastic polymeric material described herein) which consists of voids.

In some embodiments of any one of the embodiments described herein, one or more elastic layers (e.g., porous elastic layers, according to any of the respective embodiments described herein) are independently made of polymeric fibers.

Without being bound by any particular theory, it is believed that a fibrous structure of an elastic layer made of polymeric fibers advantageously allows a needle to pass through the layer by pushing fibers aside without any considerable amount of permanent deformation or mechanical disruption of the layers, and that the elasticity of the fibers causes the layers to rebound, thereby closing suture holes and holding tightly to sutures.

In some embodiments of any one of the embodiments described herein, the fibers are polymeric fibers.

The fibers which form the elastic layers may be woven or non-woven. In some embodiments of any one of the embodiments described herein, the fibers are non-woven.

In some embodiments of any one of the embodiments described herein, the fibers in the elastic layer(s) are electrospun.

Without being bound by any particular theory, it is believed that electrospun fibers, and structurally similar fibers, are particularly suitable for forming elastic layers such as described herein. In particular, layers of electrospun fibers can be prepared from a wide variety of materials, and allow control over pore size, fiber size, fiber alignment, hydrophobicity, elasticity and mechanical strength.

In some embodiments of any one of the embodiments described herein relating to polymeric fibers, at least 20 weight percent (by dry weight) of the polymeric fiber consists of one or more polymers. In some embodiments, at least 30 weight percent (by dry weight) of the polymeric fiber consists of one or more polymers. In some embodiments, at least 40 weight percent (by dry weight) of the polymeric fiber consists of one or more polymers. In some embodiments, at least 50 weight percent (by dry weight) of the polymeric fiber consists of one or more polymers. In some embodiments, at least 60 weight percent (by dry weight) of the polymeric fiber consists of one or more polymers. In some embodiments, at least 70 weight percent (by dry weight) of the polymeric fiber consists of one or more polymers. In some embodiments, at least 80 weight percent (by dry weight) of the polymeric fiber consists of one or more polymers. In some embodiments, at least 90 weight percent (by dry weight) of the polymeric fiber consists of one or more polymers. In some embodiments, the polymeric fiber consists essentially of one or more polymers.

In some embodiments of any one of the embodiments described herein, the fibers (e.g., polymeric fibers according to any of the respective embodiments described herein) in at least one of the porous layers of fibers (according to any one of the respective embodiments described herein) are characterized by a mean diameter in a range of from 0.001 to 30 μm. In some such embodiments, the mean diameter is in a range of from 0.003 to 30 μm. In some such embodiments, the mean diameter is in a range of from 0.01 to 30 μm. In some such embodiments, the mean diameter is in a range of from 0.03 to 30 μm. In some such embodiments, the mean diameter is in a range of from 0.1 to 30 μm. In some such embodiments, the mean diameter is in a range of from 0.3 to 30 μm. In some such embodiments, the mean diameter is in a range of from 1 to 10 μm. In some such embodiments, the mean diameter is in a range of from 1 to 4 μm. In some such embodiments, the mean diameter is about 3 μm.

In some embodiments of any one of the embodiments described herein, the fibers (e.g., polymeric fibers according to any of the respective embodiments described herein) in each of the porous layers of fibers (according to any one of the respective embodiments described herein) are characterized by a mean diameter in a range of from 0.001 to 30 μm. In some such embodiments, the mean diameter is in a range of from 0.003 to 30 μm. In some such embodiments, the mean diameter is in a range of from 0.01 to 30 μm. In some such embodiments, the mean diameter is in a range of from 0.03 to 30 μm. In some such embodiments, the mean diameter is in a range of from 0.1 to 30 μm. In some such embodiments, the mean diameter is in a range of from 0.3 to 30 μm. In some such embodiments, the mean diameter is in a range of from 1 to 10 μm. In some such embodiments, the mean diameter is in a range of from 1 to 4 μm. In some such embodiments, the mean diameter is about 3 μm.

In some embodiments of any one of the embodiments described herein, the fibers in at least one of the porous layers of fibers (according to any one of the respective embodiments described herein) are characterized by a mean diameter in a range of from 0.001 to 10 μm. In some such embodiments, the mean diameter is in a range of from 0.3 to 3 μm. In some such embodiments, the mean diameter is in a range of from 0.3 to 1 μm.

In some embodiments of any one of the embodiments described herein, the fibers in each of the porous layers of fibers (according to any one of the respective embodiments described herein) are characterized by a mean diameter in a range of from 0.3 to 10 μm. In some such embodiments, the mean diameter is in a range of from 0.3 to 3 μm. In some such embodiments, the mean diameter is in a range of from 0.3 to 1 μm.

In some embodiments of any one of the embodiments described herein, the fibers in at least one of the porous layers of fibers (according to any one of the respective embodiments described herein) are characterized by a mean diameter in a range of from 1 to 30 μm. In some such embodiments, the mean diameter is in a range of from 3 to 30 μm. In some such embodiments, the mean diameter is in a range of from 10 to 30 μm.

In some embodiments of any one of the embodiments described herein, the fibers in each of the porous layers of fibers (according to any one of the respective embodiments described herein) are characterized by a mean diameter in a range of from 1 to 30 μm. In some such embodiments, the mean diameter is in a range of from 3 to 30 μm. In some such embodiments, the mean diameter is in a range of from 10 to 30 μm.

In some embodiments of any one of the embodiments described herein, at least one of the elastic layers (according to any one of the respective embodiments described herein) is characterized by a mean thickness in a range of from 10 to 500 μm. In some such embodiments, the mean thickness is in a range of from 25 to 350 μm. In some such embodiments, the mean thickness is in a range of from 50 to 250 μm.

In some embodiments of any one of the embodiments described herein, each of the elastic layers (according to any one of the respective embodiments described herein) is characterized by a mean thickness in a range of from 10 to 500 μm. In some such embodiments, the mean thickness is in a range of from 25 to 350 μm. In some such embodiments, the mean thickness is in a range of from 50 to 250 μm.

In some embodiments of any one of the embodiments described herein, at least one elastic layer according to any of the respective embodiments described herein is characterized by at least one of the following 3 properties:

a) an elastic modulus (Young's modulus) in a range of from 1 kPa to 1 GPa;

b) an elongation at failure of at least 100% (e.g., in a range of from 100% to 1000%); and

c) a glass transition temperature and/or melting point of the elastic polymeric material which is at a temperature above 40° C.

Herein throughout, the phrase “elastic modulus” refers to Young's modulus, as determined by response of a material to application of tensile stress (e.g., according to procedures described in the Examples section herein).

Tensile properties described herein (e.g., elastic modulus, elongation at failure, recovery and ultimate tensile strength) are determined in accordance with ASTM international standard D882-12 for testing tensile properties of thin plastic sheeting. Except where indicated otherwise, the tensile properties are determined after the layers are immersed in aqueous liquid (e.g., water, phosphate buffer saline), and at a temperature of 37° C. (e.g., according to procedures described in the Examples section herein). Tensile testing characterizes an amount of tensile stress applied to the tested material as a function of tensile strain (increase in length due to tensile stress, as a percentage of the original length) of the material.

The ultimate tensile strength is determined as the maximal stress which can be applied to the tested material, such that any further strain is obtained with reduced stress (a phenomenon known as “necking” or is unobtainable because the tensile stress results in rupture (e.g., tearing, cracking) of the material.

The elongation at failure is determined as the maximal strain (elongation) which can occur (upon application of tensile stress equal to the ultimate tensile strength) before failure of the tested material occurs (e.g., as rupture or necking).

The elastic modulus is determined as the gradient of stress as a function of strain over ranges of stress and strain wherein stress is a linear function of strain (e.g., from a stress and strain of zero, to the elastic proportionality limit, and optionally from zero strain to a strain which is no more than 50% of the elongation at failure).

Recovery is determined by releasing the tensile stress after subjecting the tested material as the ratio of the decrease in length to a prior strain after a material (e.g., elastic layer) is subjected to a prior strain which is almost equal to the elongation at failure (optionally about 90% of the elongation at failure, optionally about 95% of the elongation at failure, optionally about 98% of the elongation at failure, optionally about 99% of the elongation at failure, wherein the elongation at failure can be determined using an equivalent sample). Thus, for example, a material extended to an elongation at failure which is 200%, and which upon release of tensile stress returns to a state characterized by a strain of 20% relative to the original length, would be characterized as having a recovery of 90% (i.e., 200%−20% divided by 200%).

In some embodiments of any one of the embodiments described herein, each of the elastic layers in a matrix according to any of the respective embodiments described herein is characterized by at least one of the abovementioned three properties.

In some embodiments of any one of the embodiments described herein, at least one elastic layer (according to any of the respective embodiments described herein) is characterized by at least two of the abovementioned three properties. In some such embodiments, each of the elastic layers in a matrix (according to any of the respective embodiments described herein) is characterized by at least two of the abovementioned three properties.

In some embodiments of any one of the embodiments described herein, at least one elastic layer (according to any of the respective embodiments described herein) is characterized by each of the abovementioned three properties. In some such embodiments, each of the elastic layers in a matrix (according to any of the respective embodiments described herein) is characterized by each of the abovementioned three properties.

In some embodiments of any one of the embodiments described herein, at least one elastic layer is characterized by a recovery of at least 75% (e.g., from 75 to 99.9%). In some such embodiments, the recovery is at least 80% (e.g., from 80 to 99.9%). In some such embodiments, the recovery is at least 85% (e.g., from 85 to 99.9%). In some such embodiments, the recovery is at least 90% (e.g., from 90 to 99.9%). In some such embodiments, the recovery is at least 95% (e.g., from 95 to 99.9%).

In some embodiments of any one of the embodiments described herein, at least one elastic layer is characterized by an elastic modulus (Young's modulus) in a range of from 1 kPa to 1 GPa. In some such embodiments, the elastic modulus is in a range of from 3 kPa to 500 MPa. In some such embodiments, the elastic modulus is in a range of from 10 kPa to 200 MPa. In some such embodiments, the elastic modulus is in a range of from 20 kPa to 100 MPa. In some such embodiments, the elastic modulus is in a range of from 50 kPa to 50 MPa. In some such embodiments, the elastic modulus is in a range of from 50 kPa to 20 MPa. In some such embodiments, the elastic modulus is in a range of from 50 kPa to 10 MPa. In some such embodiments, the elastic modulus is in a range of from 100 kPa to 3 MPa. In some such embodiments, each of the elastic layers in a matrix (according to any of the respective embodiments described herein) is characterized by an elastic modulus in a range according to any of the aforementioned embodiments.

In some embodiments of any one of the embodiments described herein, at least one elastic layer is characterized by an elastic modulus (Young's modulus) in a range of from 1 kPa to 300 MPa. In some such embodiments, the elastic modulus is in a range of from 1 kPa to 100 MPa. In some such embodiments, the elastic modulus is in a range of from 1 kPa to 30 MPa. In some such embodiments, the elastic modulus is in a range of from 1 kPa to 10 MPa. In some such embodiments, the elastic modulus is in a range of from 1 kPa to 3 MPa. In some such embodiments, the elastic modulus is in a range of from 1 kPa to 1 MPa. In some such embodiments, the elastic modulus is in a range of from 3 kPa to 1 MPa. In some such embodiments, the elastic modulus is in a range of from 10 kPa to 1 MPa. In some such embodiments, the elastic modulus is in a range of from 30 kPa to 1 MPa. In some such embodiments, each of the elastic layers in a matrix (according to any of the respective embodiments described herein) is characterized by an elastic modulus in a range according to any of the aforementioned embodiments.

In some embodiments of any one of the embodiments described herein, at least one elastic layer is characterized by an elastic modulus (Young's modulus) in a range of from 3 kPa to 1 GPa. In some such embodiments, the elastic modulus is in a range of from 10 kPa to 1 GPa. In some such embodiments, the elastic modulus is in a range of from 30 kPa to 1 GPa. In some such embodiments, the elastic modulus is in a range of from 100 kPa to 1 GPa. In some such embodiments, the elastic modulus is in a range of from 300 kPa to 1 GPa. In some such embodiments, the elastic modulus is in a range of from 300 kPa to 300 MPa. In some such embodiments, the elastic modulus is in a range of from 300 kPa to 100 MPa. In some such embodiments, the elastic modulus is in a range of from 300 kPa to 30 MPa. In some such embodiments, the elastic modulus is in a range of from 300 kPa to 10 MPa. In some such embodiments, each of the elastic layers in a core matrix (according to any of the respective embodiments described herein) is characterized by an elastic modulus in a range according to any of the aforementioned embodiments.

In some embodiments of any one of the embodiments described herein, at least one elastic layer is characterized by an elongation at failure of at least 10%. In some such embodiments, the elongation at failure is in a range of from 10% to 1000%. In some such embodiments, the elongation at failure is at least 20%. In some such embodiments, the elongation at failure is in a range of from 20% to 1000%. In some such embodiments, the elongation at failure is at least 50%. In some such embodiments, the elongation at failure is in a range of from 50% to 1000%. In some such embodiments, the elongation at failure is at least 100%. In some such embodiments, the elongation at failure is in a range of from 100% to 1000%. In some such embodiments, the elongation at failure is at least 200%. In some such embodiments, the elongation at failure is in a range of from 200% to 1000%. In some such embodiments, the elongation at failure is in a range of from 200% to 600%. In some such embodiments, each of the elastic layers in a core matrix (according to any of the respective embodiments described herein) is characterized by an elongation at failure in a range according to any of the aforementioned embodiments.

In some embodiments of any one of the embodiments described herein, at least one elastic layer is characterized by an elongation at failure of at least 10% (according to any of the respective embodiments described herein) and an elastic modulus in a range of from 1 kPa to 1 GPa (according to any of the respective embodiments described herein). In some such embodiments, each of the elastic layers in a matrix (according to any of the respective embodiments described herein) is characterized by an elongation at failure and elastic modulus in a range according to any of the aforementioned embodiments.

In some embodiments of any one of the embodiments described herein, at least one elastic layer is characterized by an elongation at failure of at least 100% (according to any of the respective embodiments described herein) and a recovery of at least 75% (according to any of the respective embodiments described herein). In some such embodiments, each of the elastic layers in a core matrix (according to any of the respective embodiments described herein) is characterized by an elongation at failure and recovery in a range according to any of the aforementioned embodiments.

In some embodiments of any one of the embodiments described herein, at least one elastic layer is characterized by an elastic modulus in a range of from 1 kPa to 1 GPa (according to any of the respective embodiments described herein) and a recovery of at least 75% (according to any of the respective embodiments described herein). In some such embodiments, each of the elastic layers in a matrix (according to any of the respective embodiments described herein) is characterized by an elastic modulus and recovery in a range according to any of the aforementioned embodiments.

In some such embodiments, the elongation at failure is at least 100%. In some such embodiments, the elongation at failure is in a range of from 100% to 1000%. In some such embodiments, the elongation at failure is at least 200%. In some such embodiments, the elongation at failure is in a range of from 200% to 1000%. In some such embodiments, the elongation at failure is in a range of from 200% to 600%. In some such embodiments, each of the elastic layers in a matrix (according to any of the respective embodiments described herein) is characterized by an elongation at failure in a range according to any of the aforementioned embodiments.

In some embodiments of any one of the embodiments described herein, at least one elastic layer is characterized by an ultimate tensile strength of at least 0.05 MPa. In some embodiments of any one of the embodiments described herein, at least one elastic layer is characterized by an ultimate tensile strength of at least 1 MPa. In some embodiments of any one of the embodiments described herein, at least one elastic layer is characterized by an ultimate tensile strength of at least 2 MPa. In some embodiments of any one of the embodiments described herein, at least one elastic layer is characterized by an ultimate tensile strength of at least 4 MPa. In some such embodiments, each of the elastic layers in a matrix (according to any of the respective embodiments described herein) is characterized by an ultimate tensile strength according to any of the aforementioned embodiments.

In some embodiments of any one of the embodiments described herein, at least one elastic layer is characterized by an ultimate tensile strength of at least 0.05 MPa, and an elongation at failure of at least 100%. In some such embodiments, the elongation at failure is in a range of from 100% to 1000%. In some such embodiments, the elongation at failure is at least 200%. In some such embodiments, the elongation at failure is in a range of from 200% to 1000%. In some such embodiments, the elongation at failure is in a range of from 200% to 600%. In some such embodiments, each of the elastic layers in a matrix (according to any of the respective embodiments described herein) is characterized by an ultimate tensile strength and elongation at failure in a range according to any of the aforementioned embodiments.

In some embodiments of any one of the embodiments described herein, at least one elastic layer is characterized by an ultimate tensile strength of at least 1 MPa, and an elongation at failure of at least 100%. In some such embodiments, the elongation at failure is in a range of from 100% to 1000%. In some such embodiments, the elongation at failure is at least 200%. In some such embodiments, the elongation at failure is in a range of from 200% to 1000%. In some such embodiments, the elongation at failure is in a range of from 200% to 600%. In some such embodiments, each of the elastic layers in a core matrix (according to any of the respective embodiments described herein) is characterized by an ultimate tensile strength and elongation at failure in a range according to any of the aforementioned embodiments.

In some embodiments of any one of the embodiments described herein, at least one elastic layer is characterized by an ultimate tensile strength of at least 2 MPa, and an elongation at failure of at least 100%. In some such embodiments, the elongation at failure is in a range of from 100% to 1000%. In some such embodiments, the elongation at failure is at least 200%. In some such embodiments, the elongation at failure is in a range of from 200% to 1000%. In some such embodiments, the elongation at failure is in a range of from 200% to 600%. In some such embodiments, each of the elastic layers in a core matrix (according to any of the respective embodiments described herein) is characterized by an ultimate tensile strength and elongation at failure in a range according to any of the aforementioned embodiments.

In some embodiments of any one of the embodiments described herein, at least one elastic layer is characterized by an ultimate tensile strength of at least 4 MPa, and an elongation at failure of at least 100%. In some such embodiments, the elongation at failure is in a range of from 100% to 1000%. In some such embodiments, the elongation at failure is at least 200%. In some such embodiments, the elongation at failure is in a range of from 200% to 1000%. In some such embodiments, the elongation at failure is in a range of from 200% to 600%. In some such embodiments, each of the elastic layers in a matrix (according to any of the respective embodiments described herein) is characterized by an ultimate tensile strength and elongation at failure in a range according to any of the aforementioned embodiments.

In most embodiments, the mechanical properties of the matrix as a whole will be strongly dependent on the mechanical properties of the elastic layer.

In some embodiments of any one of the embodiments described herein, the matrix (according to any of the respective embodiments described herein) is characterized by an elastic modulus which is within a range of 50% to 200% of an elastic modulus of at least one of the elastic layers, and optionally within a range of 50% to 200% of an elastic modulus of each of the elastic layers in the matrix. In some embodiments, the matrix elastic modulus is within a range of 80% to 120% of an elastic modulus of at least one of the elastic layers. In some embodiments, the matrix elastic modulus is within a range of 80% to 120% of an elastic modulus of each of the elastic layers in the matrix. In some embodiments of any of the aforementioned embodiments, the matrix contains one viscoelastic layer interposed between two elastic layers (according to any of the respective embodiments described herein), and the matrix elastic modulus is within a range of 50% to 200% (and optionally 80% to 120%) of an elastic modulus of at least one (optionally both) of the aforementioned two elastic layers.

The Viscoelastic Layer

In some embodiments, the layer of the viscoelastic polymeric material comprises a polymer characterized by a glass transition temperature and/or melting point at a temperature above 40° C. as further described hereinbelow. In some embodiments, each of the layers of an elastic polymeric material is independently made of polymeric fibers.

Herein, the terms “viscoelasticity” and “viscoelastic” refer to a tendency of a material (optionally in the form of a layer) to resist stress to a degree which correlates with the rate of deformation (e.g., strain, shear), at an indicated temperature or at a temperature of 37° C. (in contexts wherein no temperature is indicated). That is, when deformation is effected relatively slowly, the resistance of the material is lower (e.g., due to viscous flow during deformation), and the resistance may optionally approach zero as the rate of deformation (e.g., shear) approaches zero. The resistance will typically not be sufficient to allow the material to return to its original shape, except in some cases wherein the rate of deformation is very high.

A degree of viscoelasticity may optionally be characterized by a loss tangent (G″/G′), which is a ratio of a loss shear modulus (G″, also referred to herein interchangeably as a “shear loss modulus”) to storage shear modulus (G′, also referred to herein interchangeably as a “shear storage modulus”). A loss shear modulus reflects viscous behavior, whereas a storage shear modulus reflects elastic behavior.

In some embodiments, of any one of the embodiments described herein, a viscoelastic material (e.g., viscoelastic layer) is characterized in that a loss tangent of at least 0.01.

In some embodiments, of any one of the embodiments described herein, the viscoelastic layer is characterized by a loss tangent which is greater than a loss tangent of the elastic layer. In some embodiments, a viscoelastic layer is characterized by a loss tangent which is at least 200% of (two-fold) a loss tangent of the elastic layer.

Storage shear modulus and loss shear modulus may optionally be determined using a shear rheometer, for example, a strain-controlled rotational rheometer, at an indicated temperature and frequency (e.g., using procedures described in the Examples section herein).

The elastic and viscoelastic layers described herein are, in some embodiments thereof, made of a polymeric material selected to exhibit the elasticity and/or viscoelasticity according to any of the respective embodiments described herein. A person skilled in the art would recognize which polymeric materials (e.g., polymers and mixtures thereof) to select, and how to produce a layer therefrom in order to obtain a layer exhibiting the indicated features (e.g., elasticity and/or viscoelasticity) without undue experimentation, particularly in view of the description and guidance provided herein.

Herein, in embodiments wherein an elastic layer is made of a polymeric material, the phrase “elastic layer” and “layer of an elastic polymeric material” are used interchangeably.

Herein, in embodiments wherein a viscoelastic layer is made of a polymeric material, the phrase “viscoelastic layer” and “layer of a viscoelastic polymeric material” are used interchangeably.

In some embodiments of any one of the embodiments described herein, the matrix contains one layer of viscoelastic material.

The elastic layer(s) and viscoelastic layer(s) may be layered in any order. An elastic layer may optionally be adjacent to (e.g., in direct contact with) a viscoelastic layer and/or another elastic layer, and a viscoelastic layer may optionally be adjacent to (e.g., in direct contact with) an elastic layer and/or another viscoelastic layer. In some embodiments of any one of the embodiments described herein, the matrix comprises at least one viscoelastic layer between the elastic layers (according to any of the respective embodiments described herein). Such a configuration includes, for example, more than one viscoelastic layer between a pair of elastic layers, and one or more elastic layers which, along with the viscoelastic layer(s), are interposed between other elastic layers.

As used herein, a material (e.g., viscoelastic layer) which is “between” layers (e.g., elastic layers) is located in at least a portion of the region between the layers, and does not exclude other substances from also being between the layers, and optionally is not in contact with one or more of the layers.

Without being bound by any particular theory, it is believed that a location of a viscoelastic polymeric material between elastic layers, allows the elastic layers to contain the viscoelastic polymeric material within the matrix, and prevent significant leaching of the viscoelastic polymeric material. It is further believed that a viscoelastic polymeric material is in a form of an intermediate layer is highly suitable for acting as a barrier and for closing holes, as described herein, while being effectively contained by the elastic layers.

In some embodiments of any one of the embodiments described herein, the core matrix contains two elastic layers as described herein (according to any of the respective embodiments) and one viscoelastic layer as described herein (according to any of the respective embodiments) interposed between the two elastic layers.

Herein, the term “polymeric material” (including within the phrases “elastic polymeric material” and “viscoelastic polymeric material”) refer to a material comprising one or more polymers (as defined herein), wherein at least 20 weight percent (by dry weight) of the material consists of the one or more polymers.

In some embodiments, the term “polymer” further encompasses “oligomer”. In some embodiments of any of the embodiments described herein, at least 30 weight percent (by dry weight) of the polymeric material (e.g., elastic polymeric material and/or viscoelastic polymeric material) consists of one or more polymers. In some embodiments, at least 40 weight percent (by dry weight) of the polymeric material consists of one or more polymers. In some embodiments, at least 50 weight percent (by dry weight) of the polymeric material consists of one or more polymers. In some embodiments, at least 60 weight percent (by dry weight) of the polymeric material consists of one or more polymers. In some embodiments, at least 70 weight percent (by dry weight) of the polymeric material consists of one or more polymers. In some embodiments, at least 80 weight percent (by dry weight) of the polymeric material consists of one or more polymers. In some embodiments, at least 90 weight percent (by dry weight) of the polymeric material consists of one or more polymers. In some embodiments, the polymeric material (e.g., elastic polymeric material and/or viscoelastic polymeric material) consists essentially of one or more polymers.

The term “polymer”, as used herein, encompasses organic and inorganic polymer and further encompasses one or more of a polymer, a copolymer or a mixture thereof (a blend). Polymers used in embodiments of the invention may be synthetic and/or natural (e.g., biological) in origin.

Non-limiting examples of polymers which are suitable for use in elastic and/or viscoelastic polymeric materials described herein include homo-polymers and co-polymers such as polyesters (e.g., poly(ethylene terephthalate) and aliphatic polyesters made of glycolide (glycolic acid), lactide (lactic acid, including L-lactic acid and/or D-lactic acid), ε-caprolactone, dioxanone (e.g., p-dioxanone), trimethylene carbonate, hydroxybutyrate and/or hydroxyvalerate); polypeptides made of natural and/or modified amino acids (e.g., collagen, alginate, elastin, elastin-like polypeptides, albumin, fibrin, chitosan, silk, poly(γ-glutamic acid) and polylysine); polyethers, such as synthetic polyethers (e.g., poly(ethylene glycol)); polysaccharides made of natural and/or modified saccharides (e.g., hyaluronic acid); polydepsipeptides; biodegradable nylon co-polyamides; polydihydropyrans; polyphosphazenes; poly(orthoesters); poly(cyanoacrylates); polyanhydrides; polyurethanes; polycarbonates; silicones;

polyamides (e.g., nylons); polysulfones; polyether ether ketones (PEEKs);

polytetrafluoroethylene; polyethylene; and polyacrylate esters (e.g., poly(methyl methacrylate), poly(ethyl methacrylate), poly(methyl acrylate) and poly(ethyl acrylate)); any copolymer thereof (including any ratio of the respective monomers) and any combination thereof.

While any polymer, copolymer or a mixture of polymers and/or copolymers can be used for producing the elastic and/or viscoelastic polymeric material described herein, according to some embodiments of any one of the embodiments described herein relating to elastic and/or viscoelastic polymeric material, the elastic and/or viscoelastic polymeric material is formed of a biocompatible and/or biodegradable polymer.

In some embodiments, the elastic polymeric material, fibers formed from the elastic polymeric material and/or viscoelastic polymeric material described herein are biocompatible and biodegradable.

In some embodiments, the elastic polymeric material, fibers formed from the elastic polymeric material and/or viscoelastic polymeric material described herein are biocompatible and non-biodegradable.

As used herein, the term “biocompatible” refers to a material which the skilled practitioner would expect the body to generally accept without significant toxicity, immune response and/or rejection, or excessive fibrosis. In some embodiments, a moderate degree of immune response and/or fibrosis may optionally be acceptable or desired.

The term “biodegradable” as used in the context of the present invention, describes a material which can decompose under physiological and/or environmental conditions into breakdown products. Such physiological and/or environmental conditions include, for example, hydrolysis (decomposition via hydrolytic cleavage), enzymatic catalysis (enzymatic degradation), and mechanical interactions. This term typically refers to substances that decompose under these conditions such that 30 weight percent of the substance decompose within a time period shorter than one year.

The term “biodegradable” as used in the context of the present invention, also encompasses the term “bioresorbable”, which describes a substance that decomposes under physiological conditions to break down to products that undergo bioresorption into the host-organism, namely, become metabolites of the biochemical systems of the host-organism.

Preferred biodegradable polymers according to the present embodiments are non-toxic and benign biocompatible polymers. In some such embodiments, the biodegradable polymer is a bioresorbable polymers which decomposes into non-toxic and benign breakdown products that are absorbed in the biochemical systems of the subject.

Non-limiting examples of biodegradable polymers which are suitable for use in elastic and/or viscoelastic polymeric materials described herein include homo-polymers and co-polymers such as aliphatic polyesters made of glycolide (glycolic acid), lactide (e.g., lactic acid, including L-lactic acid and/or D-lactic acid), ε-caprolactone, dioxanone (e.g., p-dioxanone), trimethylene carbonate, hydroxybutyrate and/or hydroxyvalerate; polypeptides made of natural and/or modified amino acids (e.g., collagen, alginate, elastin, elastin-like polypeptides, albumin, fibrin, chitosan, silk, poly(γ-glutamic acid) and polylysine); polysaccharides made of natural and/or modified saccharides (e.g., hyaluronic acid); polydepsipeptides; biodegradable nylon co-polyamides; polydihydropyrans; polyphosphazenes; poly(orthoesters); poly(cyanoacrylates); polyanhydrides; copolymers thereof (including any ratio of the respective monomers); and any combination thereof.

Non-limiting examples of non-biodegradable polymers which are suitable for use in elastic and/or viscoelastic polymeric materials described herein include polyurethanes, polycarbonates, silicones, polyamides (e.g., nylons), polysulfones, polyether ether ketones (PEEKs), polytetrafluoroethylene, polyethylene, poly(methyl methacrylate), poly(ethyl methacrylate), poly(methyl acrylate), poly(ethyl acrylate) and non-biodegradable polyesters such as, for example, poly(ethylene terephthalate).

In some embodiments of any one of the embodiments described herein, any one or more of the elastic and viscoelastic layers is made of polymer fibers. In some embodiments, the fibers are electrospun fibers.

The term “fiber”, as used herein, describes a class of structural elements, similar to pieces of thread, that are made of continuous filaments and/or discrete elongated pieces.

In some embodiments of any one of the embodiments described herein, the matrix has a sheet-like geometry. In some embodiments, both the composition-of-matter and the matrix have a sheet-like geometry.

In some embodiments of any one of the embodiments described herein, the sheet-like geometry is characterized in that a mean thickness in one dimension (e.g., a mean width in the dimension in which the matrix is narrowest) is less than 20% of a mean width in each of two perpendicular dimensions. In some such embodiments, a mean thickness in one dimension is less than 10% of a mean width in each of two perpendicular dimensions. In some such embodiments, a mean thickness in one dimension is less than 5% of a mean width in each of two perpendicular dimensions. In some such embodiments, a mean thickness in one dimension is less than 2% of a mean width in each of two perpendicular dimensions. In some such embodiments, a mean thickness in one dimension is less than 1% of a mean width in each of two perpendicular dimensions. In some such embodiments, a mean thickness in one dimension is less than 0.5% of a mean width in each of two perpendicular dimensions. In some such embodiments, a mean thickness in one dimension is less than 0.2% of a mean width in each of two perpendicular dimensions. In some such embodiments, a mean thickness in one dimension is less than 0.1% of a mean width in each of two perpendicular dimensions.

In some embodiments of any one of the embodiments described herein, the matrix is characterized by a mean thickness of less than 3 mm (e.g., between 60 μm and 3 mm). In some such embodiments, the mean thickness is less than 2 mm (e.g., between 60 μm and 2 mm). In some such embodiments, the mean thickness is less than 1.5 mm (e.g., between 60 μm and 1.5 mm). In some such embodiments, the mean thickness is less than 1.25 mm (e.g., between 60 μm and 1.25 mm). In some such embodiments, the mean thickness is less than 1 mm (e.g., between 60 μm and 1 mm). In some such embodiments, the mean thickness is less than 750 μm (e.g., between 60 and 750 μm). In some such embodiments, the mean thickness is less than 500 μm (e.g., between 60 and 500 μm). In some such embodiments, the mean thickness is less than 250 μm (e.g., between 60 and 250 μm).

In some embodiments of any one of the embodiments described herein, a mean total thickness of the elastic layers is at least 50% (e.g., from 50 to 99%) of the mean thickness of the matrix. In some such embodiments, a mean total thickness of the elastic layers is at least 60% (e.g., from 60 to 99%) of the mean thickness of the matrix. In some such embodiments, a mean total thickness of the elastic layers is at least 70% (e.g., from 70 to 99%) of the mean thickness of the matrix. In some such embodiments, a mean total thickness of the elastic layers is at least 80% (e.g., from 80 to 99%) of the mean thickness of the matrix. In some such embodiments, a mean total thickness of the elastic layers is at least 90% (e.g., from 90 to 99%) of the mean thickness of the matrix.

As exemplified in the Examples section herein, multi-layer matrices as described herein exhibit a considerably degree of water-impermeability.

In some embodiments of any one of the embodiments described herein, the matrix is characterized by a water-permeability of less than 1 ml per hour per cm² upon exposure to an aqueous liquid at a pressure of 40 mmHg. In some such embodiments, the water-permeability is less than 0.3 ml per hour per cm². In some embodiments, the water-permeability is less than 0.1 ml per hour per cm². In some embodiments, the water-permeability is less than 0.03 ml per hour per cm². In some embodiments, the water-permeability is less than 0.01 ml per hour per cm².

In some embodiments of any one of the embodiments described herein, the matrix is characterized by a water-permeability of less than 1 ml per hour per cm² upon exposure to an aqueous liquid at a pressure of 15 mmHg. In some such embodiments, the water-permeability is less than 0.3 ml per hour per cm². In some embodiments, the water-permeability is less than 0.1 ml per hour per cm². In some embodiments, the water-permeability is less than 0.03 ml per hour per cm². In some embodiments, the water-permeability is less than 0.01 ml per hour per cm².

Herein, water-permeability is determined in accordance with ISO 811, according to procedures as described in the Examples section below. The matrix is placed at the bottom of a column of aqueous liquid (optionally water, and optionally phosphate buffer saline) having a height which provides the indicated pressure, at 37° C. The area of the matrix exposed to the liquid is optionally about 9 cm². The amount of aqueous liquid which passes the matrix during the course of a given period of time (optionally 30 minutes), when divided by the period of time and the area exposed to the liquid, determines the water-permeability.

In some embodiments of any one of the embodiments described herein, the composition-of-matter further comprising at least one additional ingredient (also referred to herein as “additive”) which imparts an additional functionality.

In some such embodiments, the additional ingredient(s) is in a form of at least one additional layer. The additional layer(s) is optionally on at least a portion of at least one surface of the core matrix and/or within the core matrix (e.g., between two other layers of the core matrix, as described herein).

Alternatively or additionally, in some embodiments, the additional ingredient(s) is dispersed within the core matrix and/or present on at least one surface, or a portion thereof, of the matrix.

Except where indicated otherwise, an additional ingredient is considered herein as part of the matrix when present within the core matrix, but not when present outside the matrix (e.g., on a surface or a portion of a surface of the matrix).

Examples of additional functionalities which may be imparted by an additional ingredient include, without limitation, water-impermeability, which may optionally be provided by an additive in a form of a water-impermeable layer and/or by a hydrophobic additive); inhibition of formation of an adhesion to tissue, which may be optionally be provided by an additive characterized by reduced adhesion to tissue, and/or by an agent which inhibits cell growth; reduction of risk of infection, which may optionally be provided by an antimicrobial agent, such as an antibiotic, and/or by a film which inhibits penetration of pathogens; reduction of risk of tissue rejection and/or immune response, which may optionally be provided by an agent which modulates an immune system; and adhesion to tissue without suturing, which may optionally be provided by an adhesive (e.g., applied on a surface) and/or an agent and/or surface which promotes cell growth and/or attachment (e.g., growth factors, extracellular matrix proteins, and/or other proteins). Examples of layers which may be formed from additional ingredients which impart such functionalities include, without limitation, water-impermeable layers, tissue-adhesive layers (i.e., layers characterized by enhanced adherence to cells, as compared with the core matrix without a tissue-adhesive layer), cell growth-promoting layers and anti-fouling layers (i.e., layers characterized by reduced adherence to cells, as compared with the core matrix without an anti-fouling layer).

Examples of additional ingredients which may be included in the composition-of-matter ingredient include, without limitation, adhesive materials, non-adhesive materials (e.g., materials characterize by particularly low adherence to tissue and/or other substrate), hydrophobic polymer particles, biological and/or bio-active materials, cellular components (e.g., a cell signaling protein, an extracellular matrix protein, a cell adhesion protein, a growth factor, protein A, a protease and a protease substrate), growth factors and therapeutically active agents.

Additional ingredients (e.g., therapeutically active agents) which can be beneficially incorporated into the composition-of-matter include both natural or synthetic polymeric (macro-biomolecules, for example, proteins, enzymes) and non-polymeric (small molecule therapeutics) natural or synthetic agents.

Examples of suitable therapeutically active agents include, without limitation, anti-proliferative agents, cytotoxic factors or cell cycle inhibitors, including CD inhibitors, such as p53, thymidine kinase (“TK”) and other agents useful for interfering with cell proliferation.

Examples of therapeutically active agents that inhibit cell proliferation and/or angiogenesis (antiproliferative drugs) which are particularly useful in drug-eluting systems destined for anticancer treatment, include paclitaxel, sirolimus (rapamycin), farnesylthiosalicylate (FTS, salirasib), fluoro-FTS, everolimus, zotarolimus, daunorubicin, doxorubicin, N-(5, 5-diacetoxypentyl)doxorubicin, anthracycline, mitomycin C, mitomycin A, 9-amino camptothecin, aminopertin, antinomycin, N⁸-acetyl spermidine, 1-(2-chloroethyl)-1,2-dimethanesulfonyl hydrazine, bleomycin, tallysomucin, etoposide, camptothecin, irinotecan, topotecan, 9-amino camptothecin, paclitaxel, docetaxel, esperamycin, 1,8-dihydroxy-bicyclo[7.3.1]trideca-4-ene-2,6-diyne-13-one, anguidine, morpholino-doxorubicin, vincristine, vinblastine and derivatives thereof.

Additional therapeutically active agents which can be beneficially incorporated into the composition-of-matter include antibiotic agents. Non-limiting examples of suitable antibiotic agents include gentamicin, ceftazidime, mafenide benzoyl peroxide, octopirox, erythromycin, zinc, silver, tetracyclin, triclosan, azelaic acid and its derivatives, phenoxyethanol and phenoxypropanol, ethyl acetate, clindamycin and meclocycline; sebostats such as flavinoids; alpha and beta hydroxy acids; polydiallyldimethylammonium chloride and bile salts such as scymnol sulfate and its derivatives, deoxycholate and cholate.

Additional therapeutically active agents which can be beneficially incorporated into the composition-of-matter include analgesic agents, anaesthetic agents, pain-killers, pain-reducers and the like (including NSAIDs, COX-2 inhibitors, K+ channel openers, opiates and morphinomimetics); and hemostatic agents and antihemorrhagic agents.

According to an aspect of some embodiments of the invention, there is provided a suturable and/or stapleable matrix capable of self-recovery.

Herein, the term “suturable” refers to an ability to have a needle pass through the matrix without causing a rupture (e.g., a crack or tear) in the matrix other than a localized hole similar in area to the needle cross-section.

Herein, the term “stapleable” refers to an ability to have a staple pass through the matrix without causing a rupture (e.g., a crack or tear) in the matrix other than a localized hole similar in area to the staple cross-section.

The needle and staple in the above definitions of “suturable” and “stapleable” have a cross-section (optionally, a circular cross-section) of no more than 1 mm². Optionally, the needle is a 21-gauge needle (diameter ˜0.51 mm).

Herein, the term “self-recover” refers to an ability of a material (e.g., material in the matrix) to at least partially close a hole formed in the material (optionally by a 21-gauge needle) by movement of a portion of the material into the space of the hole (e.g., by elastic rebound and/or plastic deformation), such that a hole remaining in the material the needle (if any) is less than 50% of an area of a cross-section of the object which formed the hole (e.g., optionally by a 21-gauge needle).

According to an aspect of some embodiments of the invention there is provided a multi-layer matrix comprising at least one layer of an elastic polymeric material (e.g., according to any one of the respective embodiments described herein) and at least one layer of a viscoelastic polymeric material (e.g., according to any one of the respective embodiments described herein), the matrix being characterized by a water-permeability of less than 1 ml per hour per cm² upon exposure to an aqueous liquid at a pressure of 40 mmHg (as defined herein). In some such embodiments, the matrix is a suturable matrix capable of self-recovery (e.g., according to any one of the respective embodiments described herein). Additionally, some embodiments of any of the embodiments described herein which relate to a matrix exhibit the aforementioned water-permeability.

In some embodiments of any one of the embodiments described herein, a matrix according to the aspects described herein exhibits a suture retention ability characterized in that a minimum mean force applied to a suture in the matrix which is sufficient to cause failure of the matrix is at least 100 grams force, and optionally at least 200 grams force.

Suture retention is tested based on the method described in the ANSI/AAMI/ISO 7198:1998/2001/(R) 2004 standard, as described in the Examples section below. The matrix is sutured with a single 4/0 suture (e.g., Premilene® 4/0 suture) at a minimum distance of 2 mm from its free end, and a tensile test is conducted (e.g., as described herein) in order to measure the force at failure of the matrix.

A viscoelastic polymeric material and/or viscoelastic layer according to any one of the embodiments described in this section described in this section may be combined with an elastic layer according to any one of the respective embodiments described herein.

In some embodiments of any one of the embodiments described herein relating to a viscoelastic polymeric material, the viscoelastic polymeric material comprises a polymer characterized by a glass transition temperature and/or melting point at a temperature below 50° C. In some embodiments, the polymer is characterized by a glass transition temperature and/or melting point at a temperature below 45° C. In some embodiments, the polymer is characterized by a glass transition temperature and/or melting point at a temperature below 40° C. In some embodiments, the polymer is characterized by a glass transition temperature and/or melting point at a temperature below 35° C. In some embodiments, the polymer is characterized by a glass transition temperature and/or melting point at a temperature below 30° C. In some embodiments, the polymer is characterized by a glass transition temperature and/or melting point at a temperature below 25° C. In some embodiments, the polymer is characterized by a glass transition temperature and/or melting point at a temperature below 20° C. In some embodiments, the polymer is characterized by a glass transition temperature and/or melting point at a temperature below 15° C. In some embodiments, the polymer is characterized by a glass transition temperature and/or melting point at a temperature below 10° C. In some embodiments, the polymer is characterized by a glass transition temperature and/or melting point at a temperature below 5° C. In some embodiments, the polymer is characterized by a glass transition temperature and/or melting point at a temperature below 0° C.

Herein, a glass transition temperature is preferably determined according to differential scanning calorimetry, using procedures accepted in the art for such a purpose, using cooling and heating rates of 10° C. per minute. The glass transition typically appears as an intersection between two linear regions in a plot of heat capacity as a function of temperature.

In some embodiments, the viscoelastic polymeric material comprises a polymer characterized by a glass transition temperature and/or melting point at a temperature which is at least 5° C. lower than an ambient temperature of the composition-of-matter. In some such embodiments, the glass transition temperature and/or melting point is at a temperature which is at least 10° C. lower than an ambient temperature of the composition-of-matter. In some such embodiments, glass transition temperature and/or melting point is at a temperature which is at least 20° C. lower than an ambient temperature of the composition-of-matter.

In some embodiments, the viscoelastic polymeric material comprises a polymer characterized by a glass transition temperature at a temperature which is at least 5° C. lower than an ambient temperature of the composition-of-matter. In some such embodiments, the glass transition temperature is at a temperature which is at least 10° C. lower than an ambient temperature of the composition-of-matter. In some such embodiments, glass transition temperature is at a temperature which is at least 20° C. lower than an ambient temperature of the composition-of-matter.

Herein, the phrase “ambient temperature of the composition-of-matter” generally refers to 20° C., except in the context of articles-of-manufacture comprising the composition-of-matter, in which case the phrase “ambient temperature of the composition-of-matter” refers to a temperature at which the article-of-manufacture is typically used, for example, body temperature in the context of an article-of-manufacture (e.g., medical device) for use inside a body (i.e., 37° C. for articles-of-manufacture for use inside a human body).

Without being bound by any particular theory, it is believed that for a relatively amorphous (i.e., relatively low-crystallinity) polymer, the glass transition temperature has a relatively strong effect on the rheological and mechanical properties of the polymer, whereas a melting point may be less significant and even absent. Similarly, is believed that for a relatively crystalline (i.e., relatively high-crystallinity) polymer, the melting point has a relatively strong effect on the rheological and mechanical properties of the polymer, whereas a glass transition temperature may be less significant and even absent.

In some embodiments relating to a viscoelastic polymeric material, the viscoelastic polymeric material comprises a polymer characterized by a crystallinity of at least 20%, and a melting point at a temperature below 40° C. In some such embodiments, the polymer is characterized by a melting point at a temperature below 35° C. In some such embodiments, the polymer is characterized by a melting point at a temperature below 30° C. In some such embodiments, the polymer is characterized by a melting point at a temperature below 25° C. In some embodiments, the polymer is characterized by a melting point at a temperature below 20° C. In some embodiments, the polymer is characterized by a melting point at a temperature below 15° C. In some embodiments, the polymer is characterized by a melting point at a temperature below 10° C. In some embodiments, the polymer is characterized by a melting point at a temperature below 5° C. In some embodiments, the polymer is characterized by a melting point at a temperature below 0° C.

In some embodiments, the viscoelastic polymeric material comprises a polymer characterized by a crystallinity of at least 30%, and a melting point at a temperature below 40° C. In some such embodiments, the polymer is characterized by a melting point at a temperature below 35° C. In some such embodiments, the polymer is characterized by a melting point at a temperature below 30° C. In some such embodiments, the polymer is characterized by a melting point at a temperature below 25° C. In some embodiments, the polymer is characterized by a melting point at a temperature below 20° C. In some embodiments, the polymer is characterized by a melting point at a temperature below 15° C. In some embodiments, the polymer is characterized by a melting point at a temperature below 10° C. In some embodiments, the polymer is characterized by a melting point at a temperature below 5° C. In some embodiments, the polymer is characterized by a melting point at a temperature below 0° C.

In some embodiments, the viscoelastic polymeric material comprises a polymer characterized by a crystallinity of at least 40%, and a melting point at a temperature below 40° C. In some such embodiments, the polymer is characterized by a melting point at a temperature below 35° C. In some such embodiments, the polymer is characterized by a melting point at a temperature below 30° C. In some such embodiments, the polymer is characterized by a melting point at a temperature below 25° C. In some embodiments, the polymer is characterized by a melting point at a temperature below 20° C. In some embodiments, the polymer is characterized by a melting point at a temperature below 15° C. In some embodiments, the polymer is characterized by a melting point at a temperature below 10° C. In some embodiments, the polymer is characterized by a melting point at a temperature below 5° C. In some embodiments, the polymer is characterized by a melting point at a temperature below 0° C.

In some embodiments, the viscoelastic polymeric material comprises a polymer characterized by a crystallinity of at least 50%, and a melting point at a temperature below 40° C. In some such embodiments, the polymer is characterized by a melting point at a temperature below 35° C. In some such embodiments, the polymer is characterized by a melting point at a temperature below 30° C. In some such embodiments, the polymer is characterized by a melting point at a temperature below 25° C. In some embodiments, the polymer is characterized by a melting point at a temperature below 20° C. In some embodiments, the polymer is characterized by a melting point at a temperature below 15° C. In some embodiments, the polymer is characterized by a melting point at a temperature below 10° C. In some embodiments, the polymer is characterized by a melting point at a temperature below 5° C. In some embodiments, the polymer is characterized by a melting point at a temperature below 0° C.

In some embodiments, the viscoelastic polymeric material comprises a polymer characterized by a crystallinity of at least 60%, and a melting point at a temperature below 40° C. In some such embodiments, the polymer is characterized by a melting point at a temperature below 35° C. In some such embodiments, the polymer is characterized by a melting point at a temperature below 30° C. In some such embodiments, the polymer is characterized by a melting point at a temperature below 25° C. In some embodiments, the polymer is characterized by a melting point at a temperature below 20° C. In some embodiments, the polymer is characterized by a melting point at a temperature below 15° C. In some embodiments, the polymer is characterized by a melting point at a temperature below 10° C. In some embodiments, the polymer is characterized by a melting point at a temperature below 5° C. In some embodiments, the polymer is characterized by a melting point at a temperature below 0° C.

In some embodiments, the viscoelastic polymeric material comprises a polymer characterized by a crystallinity of at least 70%, and a melting point at a temperature below 40° C. In some such embodiments, the polymer is characterized by a melting point at a temperature below 35° C. In some such embodiments, the polymer is characterized by a melting point at a temperature below 30° C. In some such embodiments, the polymer is characterized by a melting point at a temperature below 25° C. In some embodiments, the polymer is characterized by a melting point at a temperature below 20° C. In some embodiments, the polymer is characterized by a melting point at a temperature below 15° C. In some embodiments, the polymer is characterized by a melting point at a temperature below 10° C. In some embodiments, the polymer is characterized by a melting point at a temperature below 5° C. In some embodiments, the polymer is characterized by a melting point at a temperature below 0° C.

In some embodiments, the viscoelastic polymeric material comprises a polymer characterized by a crystallinity of at least 80%, and a melting point at a temperature below 40° C. In some such embodiments, the polymer is characterized by a melting point at a temperature below 35° C. In some such embodiments, the polymer is characterized by a melting point at a temperature below 30° C. In some such embodiments, the polymer is characterized by a melting point at a temperature below 25° C. In some embodiments, the polymer is characterized by a melting point at a temperature below 20° C. In some embodiments, the polymer is characterized by a melting point at a temperature below 15° C. In some embodiments, the polymer is characterized by a melting point at a temperature below 10° C. In some embodiments, the polymer is characterized by a melting point at a temperature below 5° C. In some embodiments, the polymer is characterized by a melting point at a temperature below 0° C.

In some embodiments, the viscoelastic polymeric material comprises a polymer characterized by a crystallinity of less than 80%, and a glass transition temperature at a temperature below 40° C. In some such embodiments, the polymer is characterized by a glass transition temperature at a temperature below 35° C. In some such embodiments, the polymer is characterized by a glass transition temperature at a temperature below 30° C. In some such embodiments, the polymer is characterized by a glass transition temperature at a temperature below 25° C. In some embodiments, the polymer is characterized by a glass transition temperature at a temperature below 20° C. In some embodiments, the polymer is characterized by a glass transition temperature at a temperature below 15° C. In some embodiments, the polymer is characterized by a glass transition temperature at a temperature below 10° C. In some embodiments, the polymer is characterized by a glass transition temperature at a temperature below 5° C. In some embodiments, the polymer is characterized by a glass transition temperature at a temperature below 0° C.

In some embodiments, the viscoelastic polymeric material comprises a polymer characterized by a crystallinity of less than 70%, and a glass transition temperature at a temperature below 40° C. In some such embodiments, the polymer is characterized by a glass transition temperature at a temperature below 35° C. In some such embodiments, the polymer is characterized by a glass transition temperature at a temperature below 30° C. In some such embodiments, the polymer is characterized by a glass transition temperature at a temperature below 25° C. In some embodiments, the polymer is characterized by a glass transition temperature at a temperature below 20° C. In some embodiments, the polymer is characterized by a glass transition temperature at a temperature below 15° C. In some embodiments, the polymer is characterized by a glass transition temperature at a temperature below 10° C. In some embodiments, the polymer is characterized by a glass transition temperature at a temperature below 5° C. In some embodiments, the polymer is characterized by a glass transition temperature at a temperature below 0° C.

In some embodiments, the viscoelastic polymeric material comprises a polymer characterized by a crystallinity of less than 60%, and a glass transition temperature at a temperature below 40° C. In some such embodiments, the polymer is characterized by a glass transition temperature at a temperature below 35° C. In some such embodiments, the polymer is characterized by a glass transition temperature at a temperature below 30° C. In some such embodiments, the polymer is characterized by a glass transition temperature at a temperature below 25° C. In some embodiments, the polymer is characterized by a glass transition temperature at a temperature below 20° C. In some embodiments, the polymer is characterized by a glass transition temperature at a temperature below 15° C. In some embodiments, the polymer is characterized by a glass transition temperature at a temperature below 10° C. In some embodiments, the polymer is characterized by a glass transition temperature at a temperature below 5° C. In some embodiments, the polymer is characterized by a glass transition temperature at a temperature below 0° C.

In some embodiments, the viscoelastic polymeric material comprises a polymer characterized by a crystallinity of less than 50%, and a glass transition temperature at a temperature below 40° C. In some such embodiments, the polymer is characterized by a glass transition temperature at a temperature below 35° C. In some such embodiments, the polymer is characterized by a glass transition temperature at a temperature below 30° C. In some such embodiments, the polymer is characterized by a glass transition temperature at a temperature below 25° C. In some embodiments, the polymer is characterized by a glass transition temperature at a temperature below 20° C. In some embodiments, the polymer is characterized by a glass transition temperature at a temperature below 15° C. In some embodiments, the polymer is characterized by a glass transition temperature at a temperature below 10° C. In some embodiments, the polymer is characterized by a glass transition temperature at a temperature below 5° C. In some embodiments, the polymer is characterized by a glass transition temperature at a temperature below 0° C.

In some embodiments, the viscoelastic polymeric material comprises a polymer characterized by a crystallinity of less than 40%, and a glass transition temperature at a temperature below 40° C. In some such embodiments, the polymer is characterized by a glass transition temperature at a temperature below 35° C. In some such embodiments, the polymer is characterized by a glass transition temperature at a temperature below 30° C. In some such embodiments, the polymer is characterized by a glass transition temperature at a temperature below 25° C. In some embodiments, the polymer is characterized by a glass transition temperature at a temperature below 20° C. In some embodiments, the polymer is characterized by a glass transition temperature at a temperature below 15° C. In some embodiments, the polymer is characterized by a glass transition temperature at a temperature below 10° C. In some embodiments, the polymer is characterized by a glass transition temperature at a temperature below 5° C. In some embodiments, the polymer is characterized by a glass transition temperature at a temperature below 0° C.

In some embodiments, the viscoelastic polymeric material comprises a polymer characterized by a crystallinity of less than 30%, and a glass transition temperature at a temperature below 40° C. In some such embodiments, the polymer is characterized by a glass transition temperature at a temperature below 35° C. In some such embodiments, the polymer is characterized by a glass transition temperature at a temperature below 30° C. In some such embodiments, the polymer is characterized by a glass transition temperature at a temperature below 25° C. In some embodiments, the polymer is characterized by a glass transition temperature at a temperature below 20° C. In some embodiments, the polymer is characterized by a glass transition temperature at a temperature below 15° C. In some embodiments, the polymer is characterized by a glass transition temperature at a temperature below 10° C. In some embodiments, the polymer is characterized by a glass transition temperature at a temperature below 5° C. In some embodiments, the polymer is characterized by a glass transition temperature at a temperature below 0° C.

In some embodiments, the viscoelastic polymeric material comprises a polymer characterized by a crystallinity of less than 20%, and a glass transition temperature at a temperature below 40° C. In some such embodiments, the polymer is characterized by a glass transition temperature at a temperature below 35° C. In some such embodiments, the polymer is characterized by a glass transition temperature at a temperature below 30° C. In some such embodiments, the polymer is characterized by a glass transition temperature at a temperature below 25° C. In some embodiments, the polymer is characterized by a glass transition temperature at a temperature below 20° C. In some embodiments, the polymer is characterized by a glass transition temperature at a temperature below 15° C. In some embodiments, the polymer is characterized by a glass transition temperature at a temperature below 10° C. In some embodiments, the polymer is characterized by a glass transition temperature at a temperature below 5° C. In some embodiments, the polymer is characterized by a glass transition temperature at a temperature below 0° C.

In some embodiments, the viscoelastic polymeric material comprises a polymer characterized by a crystallinity of less than 10%, and a glass transition temperature at a temperature below 40° C. In some such embodiments, the polymer is characterized by a glass transition temperature at a temperature below 35° C. In some such embodiments, the polymer is characterized by a glass transition temperature at a temperature below 30° C. In some such embodiments, the polymer is characterized by a glass transition temperature at a temperature below 25° C. In some embodiments, the polymer is characterized by a glass transition temperature at a temperature below 20° C. In some embodiments, the polymer is characterized by a glass transition temperature at a temperature below 15° C. In some embodiments, the polymer is characterized by a glass transition temperature at a temperature below 10° C. In some embodiments, the polymer is characterized by a glass transition temperature at a temperature below 5° C. In some embodiments, the polymer is characterized by a glass transition temperature at a temperature below 0° C.

In some embodiments, the viscoelastic polymeric material comprises (and optionally consists essentially of) one or more polymers which are biocompatible and/or biodegradable (as defined herein).

Poly(lactic acid-co-ε-caprolactone) (optionally poly(DL-lactic acid-co-ε-caprolactone, either alone or in combination with poly(L-lactic acid-co-ε-caprolactone) and/or poly(D-lactic acid-co-ε-caprolactone)) is an exemplary biocompatible and biodegradable polymer, which may be included in a viscoelastic polymeric material according to any of the respective embodiments described herein. In some such embodiments, the viscoelastic polymeric material consists essentially of poly(lactic acid-co-ε-caprolactone).

In some embodiments, the viscoelastic polymeric material comprises (and optionally consists essentially of) any one or more of the polymers and/or copolymers described herein for use in an elastic layer.

The skilled practitioner will be readily capable of selecting concentrations of polymers, molecular weights of polymers and/or molar ratios of monomers (e.g., lactic acid and ε-caprolactone) in copolymers which may provide elastic or viscoelastic properties according to any of the respective embodiments described herein relating to elastic and/or viscoelastic polymeric materials.

In some embodiments, the viscoelastic polymeric material comprises (and optionally consists essentially of) one or more hydrophobic polymers.

Without being bound by any particular theory, it is believed that a hydrophobic polymer may considerably reduce water-permeability of the matrix, even in embodiments in which the viscoelastic polymeric layer is not in a form of a continuous film. For example, pores in a porous hydrophobic viscoelastic polymeric layer may be too small to allow passage of water, as contact between the water and hydrophobic polymer is energetically unfavorable.

Herein, a “hydrophobic polymer” is a polymer characterized in that in water at a pH of 7.0, the polymer (in bulk) has a solubility of less than 1 gram per liter, and does not absorb more than 20 weight percents of water (weight of absorbed water relative to weight of polymer). In some embodiments, the hydrophobic polymer is characterized in that it does not absorb more than 10 weight percents of water at pH 7.0. In some embodiments, the hydrophobic polymeric substance is characterized in that it does not absorb more than 5 weight percents of water at pH 7.0. In some embodiments, the hydrophobic polymeric substance is characterized in that it does not absorb more than 2 weight percents of water at pH 7.0. In some embodiments, the hydrophobic polymeric substance is characterized in that it does not absorb more than 1 weight percents of water at pH 7.0.

The skilled practitioner will be readily capable of selecting polymers (e.g., polymers described herein), molecular weights of polymers and/or molar ratios of monomers (e.g., lactic acid and ε-caprolactone) in copolymers which result in a hydrophobic polymer as defined herein.

In some embodiments, a viscoelastic layer is characterized by a mean thickness in a range of from 1 to 300 μm. In some such embodiments, the mean thickness is in a range of from 2 to 250 μm. In some such embodiments, the mean thickness is in a range of from 3 to 200 μm. In some such embodiments, the mean thickness is in a range of from 5 to 150 μm. In some such embodiments, the mean thickness is in a range of from 10 to 100 μm. In some such embodiments, the mean thickness is in a range of from 15 to 60 μm.

In some embodiments, the viscoelastic layer is characterized by a mean thickness in a range of from 1 to 200 μm. In some such embodiments, the mean thickness is in a range of from 1 to 100 μm. In some such embodiments, the mean thickness is in a range of from 1 to 60 μm. In some such embodiments, the mean thickness is in a range of from 1 to 30 μm.

In some embodiments, the viscoelastic layer is characterized by a mean thickness in a range of from 2 to 300 μm. In some such embodiments, the mean thickness is in a range of from 5 to 300 μm. In some such embodiments, the mean thickness is in a range of from 10 to 300 μm. In some such embodiments, the mean thickness is in a range of from 20 to 300 μm. In some such embodiments, the mean thickness is in a range of from 40 to 300 μm.

In some embodiments, the viscoelastic layer is a non-porous, continuous film or is characterized by a limited porosity.

In some embodiments, the viscoelastic layer is characterized by a porosity which is lower than a porosity of each of the adjacent elastic layers (according to any of the respective embodiments described herein). In some such embodiments, the viscoelastic layer is characterized by a porosity which is less than 75% of a porosity of each of the adjacent elastic layers (according to any of the respective embodiments described herein). In some such embodiments, the viscoelastic layer is characterized by a porosity which is less than 50% of a porosity of each of the adjacent elastic layers (according to any of the respective embodiments described herein). In some such embodiments, the viscoelastic layer is characterized by a porosity which is less than 25% of a porosity of each of the adjacent elastic layers (according to any of the respective embodiments described herein). In some such embodiments, the viscoelastic layer is characterized by a porosity which is less than 15% of a porosity of each of the adjacent elastic layers (according to any of the respective embodiments described herein). In some such embodiments, the viscoelastic layer is characterized by a porosity which is less than 10% of a porosity of each of the adjacent elastic layers (according to any of the respective embodiments described herein). In some such embodiments, the viscoelastic layer is characterized by a porosity which is less than 5% of a porosity of each of the adjacent elastic layers (according to any of the respective embodiments described herein). In some embodiments of any one of the aforementioned embodiments relating to porosity of the viscoelastic layer(s), the elastic layers are characterized by a porosity of at least 50% (e.g., from 50 to 99%), according to any of the respective embodiments described herein.

In some embodiments, a viscoelastic layer is characterized by a porosity in a range of from 0 to 50%. In some such embodiments, the porosity is from 0 to 40%. In some such embodiments, the porosity is from 0 to 30%. In some such embodiments, the porosity is from 0 to 20%. In some such embodiments, the porosity is from 0 to 10%. In some such embodiments, a porosity of each of the adjacent elastic layers is higher than the porosity of the viscoelastic layer (e.g., more than 50%).

Without being bound by any particular theory, it is believed that a viscoelastic layer which is non-porous or characterized by limited porosity (e.g., up to 50%) reduces a permeability of the core matrix to water as well as other liquids, thereby enhancing the ability of the composition-of-matter to serve, for example, as a sealant against fluid leakage. It is further believed that such a layer, for example, a layer which does not have any fibrous structure, can readily undergo deformation in response to stress by viscous flow, and that such deformation can result in closure of holes formed in the viscoelastic layer.

In some embodiments, the viscoelastic layer is characterized by a porosity (e.g., up to 50%) which is lower than a porosity of the elastic layers (according to any of the respective embodiments described herein, optionally embodiments wherein a porosity of the elastic layers is at least 50%, at least 60%, at least 70%, at least 80% and/or at least 90%). In some such embodiments, the viscoelastic layer porosity is no more than half of the elastic layer porosity.

Without being bound by any particular theory, it is believed that the viscoelastic layer acts as a barrier (e.g., to water-permeation), which may be more impermeable than elastic layers which are more porous than the viscoelastic layer (e.g., porous elastic layers made of fibers), thereby significantly reducing permeability of matrices comprising such elastic layers.

A continuous film may optionally be prepared, for example, by film casting (e.g., as exemplified herein).

A limited porosity may optionally be prepared, for example, by forming fibers of the viscoelastic polymeric material, for example, by electrospinning (e.g., as exemplified herein), wherein the fibers partially merge as a result of viscous flow (which is optionally enhanced by heat treatment and/or pressure), thereby resulting in smaller pores and lower porosity.

In some embodiments, the viscoelastic layer has a fibrous structure. In some such embodiments, the layer comprises fibers which provide mechanical strength, as well as viscoelastic polymeric material in the spaces interposed between the fibers. In some such embodiments, the fibers are more elastic and less fluid than the viscoelastic polymeric material in the spaces interposed between the fibers. For example, in some embodiments, a relatively fluid fraction of the viscoelastic polymeric material exits the fibers by viscous flow, whereas the fraction of the viscoelastic polymeric material remaining in the fibers is more solid and/or elastic in nature.

In some embodiments, the viscoelastic layer is characterized by at least one of the following 4 properties: a) a shear storage modulus (G′) in a range of from 0.01 to 10 MPa, at a temperature of 10° C. and frequency of 0.1 Hz; b) a shear loss modulus (G″) in a range of from 0.0001 to 2 MPa, at a temperature of 10° C. and frequency of 0.1 Hz; c) a glass transition temperature and/or melting point of the viscoelastic polymeric material which is at a temperature below 40° C.; and d) a loss tangent (G″/G′) at a temperature of 10° C. and frequency of 0.1 Hz which is in a range of from 0.01 to 4.

In some embodiments, the viscoelastic layer is characterized by at least two, at least three, or each of the abovementioned 4 properties.

In some embodiments, the viscoelastic layer is characterized by a shear storage modulus (G′) in a range of from 0.01 to 10 MPa, at a temperature of 10° C. and frequency of 0.1 Hz. In some such embodiments, the shear storage modulus is in a range of from 0.05 to 10 MPa. In some such embodiments, the shear storage modulus is in a range of from 0.1 to 5 MPa. In some such embodiments, the shear storage modulus is in a range of from 0.2 to 2.5 MPa.

In some embodiments, the viscoelastic layer is characterized by a shear storage modulus (G′) in a range of from 0.01 to 1 MPa, at a temperature of 10° C. and frequency of 0.1 Hz. In some such embodiments, the shear storage modulus is in a range of from 0.05 to 1 MPa. In some such embodiments, the shear storage modulus is in a range of from 0.1 to 1 MPa. In some such embodiments, the shear storage modulus is in a range of from 0.2 to 1 MPa.

In some embodiments, the viscoelastic layer is characterized by a shear storage modulus (G′) in a range of from 0.5 to 10 MPa, at a temperature of 10° C. and frequency of 0.1 Hz. In some such embodiments, the shear storage modulus is in a range of from 1 to 10 MPa. In some such embodiments, the shear storage modulus is in a range of from 2 to 10 MPa.

In some embodiments, the viscoelastic layer is characterized by a shear loss modulus (G″) in a range of from 0.0001 to 2 MPa, at a temperature of 10° C. and frequency of 0.1 Hz. In some such embodiments, the shear loss modulus is in a range of from 0.0003 to 0.3 MPa. In some such embodiments, the shear loss modulus is in a range of from 0.001 to 0.1 MPa. In some such embodiments, the shear loss modulus is in a range of from 0.003 to 0.03 MPa.

In some embodiments, the viscoelastic layer is characterized by a shear loss modulus (G″) in a range of from 0.0001 to 0.3 MPa, at a temperature of 10° C. and frequency of 0.1 Hz. In some such embodiments, the shear loss modulus is in a range of from 0.0001 to 0.1 MPa. In some such embodiments, the shear loss modulus is in a range of from 0.0001 to 0.03 MPa. In some such embodiments, the shear loss modulus is in a range of from 0.0001 to 0.01 MPa. In some such embodiments, the shear loss modulus is in a range of from 0.0001 to 0.003 MPa. In some such embodiments, the shear loss modulus is in a range of from 0.0001 to 0.001 MPa.

In some embodiments, the viscoelastic layer is characterized by a shear loss modulus (G″) in a range of from 0.0003 to 2 MPa, at a temperature of 10° C. and frequency of 0.1 Hz. In some such embodiments, the shear loss modulus is in a range of from 0.001 to 1 MPa. In some such embodiments, the shear loss modulus is in a range of from 0.003 to 1 MPa. In some such embodiments, the shear loss modulus is in a range of from 0.01 to 1 MPa. In some such embodiments, the shear loss modulus is in a range of from 0.03 to 1 MPa. In some such embodiments, the shear loss modulus is in a range of from 0.1 to 1 MPa.

In some embodiments, the viscoelastic layer is characterized by a loss tangent (G″/G′, e.g., wherein values of G″ and G′ are each individually in accordance with any of the respective embodiments described herein) in a range of from 0.01 to 1, at a temperature of 10° C. and frequency of 0.1 Hz. In some such embodiments, the loss tangent is in a range of from 0.02 to 0.8. In some such embodiments, the loss tangent is in a range of from 0.05 to 0.7. In some such embodiments, the loss tangent is in a range of from 0.1 to 0.6. In some such embodiments, the loss tangent is in a range of from 0.175 to 0.5.

In some embodiments, the viscoelastic layer is characterized by a loss tangent (G″/G′, e.g., wherein values of G″ and G′ are each individually in accordance with any of the respective embodiments described herein) in a range of from 0.01 to 0.5, at a temperature of 10° C. and frequency of 0.1 Hz. In some such embodiments, the loss tangent is in a range of from 0.01 to 0.3. In some such embodiments, the loss tangent is in a range of from 0.01 to 0.2. In some such embodiments, the loss tangent is in a range of from 0.01 to 0.1.

In some embodiments, the viscoelastic layer is characterized by a loss tangent (G″/G′, e.g., wherein values of G″ and G′ are each individually in accordance with any of the respective embodiments described herein) in a range of from 0.02 to 1, at a temperature of 10° C. and frequency of 0.1 Hz. In some such embodiments, the loss tangent is in a range of from 0.05 to 1. In some such embodiments, the loss tangent is in a range of from 0.1 to 1. In some such embodiments, the loss tangent is in a range of from 0.2 to 1. In some such embodiments, the loss tangent is in a range of from 0.3 to 1. In some such embodiments, the loss tangent is in a range of from 0.5 to 1.

In some embodiments, the viscoelastic layer is characterized by a loss tangent (G″/G′) in a range of from 0.01 to 1 (according to any of the respective embodiments described herein), and a shear storage modulus (G′) in a range of from 0.01 to 10 MPa (according to any of the respective embodiments described herein), at a temperature of 10° C. and frequency of 0.1 Hz. In some such embodiments, the shear storage modulus is in a range of from 0.05 to 10 MPa. In some such embodiments, the shear storage modulus is in a range of from 0.1 to 5 MPa. In some such embodiments, the shear storage modulus is in a range of from 0.2 to 2.5 MPa.

In some embodiments, the viscoelastic layer is characterized by a loss tangent (G″/G′) in a range of from 0.05 to 0.7 (according to any of the respective embodiments described herein), and a shear storage modulus (G′) in a range of from 0.01 to 10 MPa (according to any of the respective embodiments described herein), at a temperature of 10° C. and frequency of 0.1 Hz. In some such embodiments, the shear storage modulus is in a range of from 0.05 to 10 MPa. In some such embodiments, the shear storage modulus is in a range of from 0.1 to 5 MPa. In some such embodiments, the shear storage modulus is in a range of from 0.2 to 2.5 MPa.

In some embodiments, the viscoelastic layer is characterized by a loss tangent (G″/G′) in a range of from 0.175 to 0.5 (according to any of the respective embodiments described herein), and a shear storage modulus (G′) in a range of from 0.01 to 10 MPa (according to any of the respective embodiments described herein), at a temperature of 10° C. and frequency of 0.1 Hz. In some such embodiments, the shear storage modulus is in a range of from 0.05 to 10 MPa. In some such embodiments, the shear storage modulus is in a range of from 0.1 to 5 MPa. In some such embodiments, the shear storage modulus is in a range of from 0.2 to 2.5 MPa.

In some embodiments, the viscoelastic layer is characterized by a loss tangent (G″/G′) in a range of from 0.01 to 1 (according to any of the respective embodiments described herein), and a shear loss modulus (G″) in a range of from 0.0001 to 2 MPa (according to any of the respective embodiments described herein), at a temperature of 10° C. and frequency of 0.1 Hz. In some such embodiments, the shear loss modulus is in a range of from 0.0003 to 0.3 MPa. In some such embodiments, the shear loss modulus is in a range of from 0.001 to 0.1 MPa. In some such embodiments, the shear loss modulus is in a range of from 0.003 to 0.03 MPa.

In some embodiments, the viscoelastic layer is characterized by a loss tangent (G″/G′) in a range of from 0.05 to 0.7 (according to any of the respective embodiments described herein), and a shear loss modulus (G″) in a range of from 0.0001 to 2 MPa (according to any of the respective embodiments described herein), at a temperature of 10° C. and frequency of 0.1 Hz. In some such embodiments, the shear loss modulus is in a range of from 0.0003 to 0.3 MPa. In some such embodiments, the shear loss modulus is in a range of from 0.001 to 0.1 MPa. In some such embodiments, the shear loss modulus is in a range of from 0.003 to 0.03 MPa.

In some embodiments, the viscoelastic layer is characterized by a loss tangent (G″/G′) in a range of from 0.175 to 0.5 (according to any of the respective embodiments described herein), and a shear loss modulus (G″) in a range of from 0.0001 to 2 MPa (according to any of the respective embodiments described herein), at a temperature of 10° C. and frequency of 0.1 Hz. In some such embodiments, the shear loss modulus is in a range of from 0.0003 to 0.3 MPa. In some such embodiments, the shear loss modulus is in a range of from 0.001 to 0.1 MPa. In some such embodiments, the shear loss modulus is in a range of from 0.003 to 0.03 MPa.

In some embodiments, the viscoelastic layer is characterized by a shear storage modulus (G′) in a range of from 0.01 to 10 MPa (according to any of the respective embodiments described herein), and a shear loss modulus (G″) in a range of from 0.0001 to 2 MPa (according to any of the respective embodiments described herein), at a temperature of 10° C. and frequency of 0.1 Hz. In some such embodiments, the shear loss modulus is in a range of from 0.0003 to 0.3 MPa. In some such embodiments, the shear loss modulus is in a range of from 0.001 to 0.1 MPa. In some such embodiments, the shear loss modulus is in a range of from 0.003 to 0.03 MPa.

In some embodiments, the viscoelastic layer is characterized by a shear storage modulus (G′) in a range of from 0.05 to 10 MPa (according to any of the respective embodiments described herein), and a shear loss modulus (G″) in a range of from 0.0001 to 2 MPa (according to any of the respective embodiments described herein), at a temperature of 10° C. and frequency of 0.1 Hz. In some such embodiments, the shear loss modulus is in a range of from 0.0003 to 0.3 MPa. In some such embodiments, the shear loss modulus is in a range of from 0.001 to 0.1 MPa. In some such embodiments, the shear loss modulus is in a range of from 0.003 to 0.03 MPa.

In some embodiments, the viscoelastic layer is characterized by a shear storage modulus (G′) in a range of from 0.1 to 5 MPa (according to any of the respective embodiments described herein), and a shear loss modulus (G″) in a range of from 0.0001 to 2 MPa (according to any of the respective embodiments described herein), at a temperature of 10° C. and frequency of 0.1 Hz. In some such embodiments, the shear loss modulus is in a range of from 0.0003 to 0.3 MPa. In some such embodiments, the shear loss modulus is in a range of from 0.001 to 0.1 MPa. In some such embodiments, the shear loss modulus is in a range of from 0.003 to 0.03 MPa.

In some embodiments, the viscoelastic layer is characterized by a shear storage modulus (G′) in a range of from 0.2 to 2.5 MPa (according to any of the respective embodiments described herein), and a shear loss modulus (G″) in a range of from 0.0001 to 2 MPa (according to any of the respective embodiments described herein), at a temperature of 10° C. and frequency of 0.1 Hz. In some such embodiments, the shear loss modulus is in a range of from 0.0003 to 0.3 MPa. In some such embodiments, the shear loss modulus is in a range of from 0.001 to 0.1 MPa. In some such embodiments, the shear loss modulus is in a range of from 0.003 to 0.03 MPa.

In some embodiments, the viscoelastic layer is characterized by a loss tangent (G″/G′) which is at least 200% of (two-fold) a loss tangent of the elastic layers (according to any of the respective embodiments described herein), at a frequency of 0.1 Hz, and at any temperature within the range of from 0 to 40° C. In some such embodiments, the temperature is 0 to 37° C. In some such embodiments, the temperature is 0 to 25° C. In some such embodiments, the temperature is 0 to 20° C. In some such embodiments, the temperature is 0° C.

In some embodiments, the viscoelastic layer is characterized by a loss tangent (G″/G′) which is at least 300% of (3-fold) a loss tangent of the elastic layers (according to any of the respective embodiments described herein), at a frequency of 0.1 Hz, and at any temperature within the range of from 0 to 40° C. In some such embodiments, the temperature is 0 to 37° C. In some such embodiments, the temperature is 0 to 25° C. In some such embodiments, the temperature is 0 to 20° C. In some such embodiments, the temperature is 0° C.

In some embodiments, the viscoelastic layer is characterized by a loss tangent (G″/G′) which is at least 500% of (5-fold) a loss tangent of the elastic layers (according to any of the respective embodiments described herein), at a frequency of 0.1 Hz, and at any temperature within the range of from 0 to 40° C. In some such embodiments, the temperature is 0 to 37° C. In some such embodiments, the temperature is 0 to 25° C. In some such embodiments, the temperature is 0 to 20° C. In some such embodiments, the temperature is 0° C.

In some embodiments, the viscoelastic layer is characterized by a loss tangent (G″/G′) which is at least 1,000% of (10-fold) a tangent of the elastic layers (according to any of the respective embodiments described herein), at a frequency of 0.1 Hz, and at any temperature within the range of from 0 to 40° C. In some such embodiments, the temperature is 0 to 37° C. In some such embodiments, the temperature is 0 to 25° C. In some such embodiments, the temperature is 0 to 20° C. In some such embodiments, the temperature is 0° C.

In some embodiments, the viscoelastic layer is characterized by a loss tangent (G″/G′) which is at least 3,000% of (30-fold) a loss tangent of the elastic layers (according to any of the respective embodiments described herein), at a frequency of 0.1 Hz, and at any temperature within the range of from 0 to 40° C. In some such embodiments, the temperature is 0 to 37° C. In some such embodiments, the temperature is 0 to 25° C. In some such embodiments, the temperature is 0 to 20° C. In some such embodiments, the temperature is 0° C.

In some embodiments, the viscoelastic layer is characterized by a loss tangent (G″/G′) which is at least 10,000% of (100-fold) a loss tangent of the elastic layers (according to any of the respective embodiments described herein), at a frequency of 0.1 Hz, and at any temperature within the range of from 0 to 40° C. In some such embodiments, the temperature is 0 to 37° C. In some such embodiments, the temperature is 0 to 25° C. In some such embodiments, the temperature is 0 to 20° C. In some such embodiments, the temperature is 0° C.

Without being bound by any particular theory, it is believed that a core matrix wherein the viscoelastic layer has a considerably higher loss tangent (and accordingly, a less solid behavior) than the elastic layers may undergo an elastic deformation in which the viscoelastic polymeric material may concomitantly undergo non-elastic deformation and viscous flow within the core matrix, while the matrix retains elastic properties due to the elastic properties of the elastic layers.

The Adhesive Layer of the Matrix:

As described hereinthroughout, the matrix may also comprise a polymerizable composition for forming adhesive materials (also referred to as “adhesives”), thus forming an additional layer or layers e.g., a polymerizable resin selected from a polymerizable monomer. The adhesive material may optionally comprise filler. In some embodiments, the composition comprises an adhesion promoter.

The strength of the adhesiveness may be dependent, inter alia, on chemical and Van der Waals interfacial forces between the polymer matrix and filler particles. These forces may be enhanced by the presence of polar functional groups on the polymer and/or by the treatment of filler surfaces with silanes, titanates, or other surface-active agents.

In some embodiments, the composition comprises at least one polymerization initiator, sensibilizer or stabilizer. These compositions may yield materials with a shrinkage of less than 1.5% when polymerized or with a shrinkage e.g., at the range of 1.5% to 3% when polymerized. The composition may comprise up to 70 wt. % of filler and becomes flowable under pressure and/or shear stress. The disclosed composition demonstrates compressive strength of e.g., around only 170 MPa and rather high values of shrinkage.

In some embodiments, the adhesive material is attached in/on at least one portion of one or more of the elastic layer. In some embodiments, the term “portion” should be construed as meaning part (e.g., 10%, 20%, 30%, or 40%) or the entire surface (e.g., the upper layer) of the one or more elastic materials. In another embodiment, the term “portion” refers to the upper surface.

In another embodiment, the adhesive material is in the form of a layer or coating. The layer or coating may be in the form of a thin thickness of e.g., 0.01 mm, 0.05 mm, 0.1 mm, 0.5 mm, 1 mm, 2 mm, or 3 mm, including any value and range therebetween. Non-limiting exemplary adhesives are selected from acrylates e.g., triethylene glycol dimethacrylate, and urethane dimethacrylate.

In some embodiments, the polymerizable monomer is selected from, without being limited thereto, mono- and multifunctional acrylates or methacrylates, e.g., methyl methacrylate, triethylene glycol dimethacrylate, 2-hydroxyethyl methacrylate, hexanediol methacrylate, or dodecanediol dimethacrylate.

In some embodiments, the “monomer” refers to substantially one type of monomer. In some embodiments, the monomer is present as part of a mixture of different monomers. In some embodiments, the monomer is polymerizable by free radical polymerization.

In some embodiments, the free radical polymerization may be initiated by irradiation. In some embodiments, the free radical polymerization may be initiated by visible light irradiation. In some embodiments, the free radical polymerization may be initiated by irradiation in the ultra-violet (UV) or near UV spectrum. In some embodiments, the free radical polymerization may be initiated by irradiation in the infra-red (IR) or near IR spectrum. For example, in some embodiments, the free radical polymerization may be initiated by irradiation in the in the range of e.g., 250 nm to 600 nm.

In some embodiments, light source used for polymerization as described hereinthroughout, may be a source of electromagnetic radiation, for example, actinic light. In alternative embodiments, curing mechanism is heat, pressure, or combination thereof.

In some embodiments, the composition also comprises a reducing initiator selected from tertiary amines. Reducing initiators may be used as reducing agents in combination with oxidizing initiators such as benzoyl peroxide, lauryl peroxide, or α-diketones, to affect more rapid generation of radicals. Non-limiting exemplary reducing initiators for self-cured polymerization are N,N-dimethyl-p-toluidine and N,N-dimethyl-sym-xylidine. Further exemplary reducing initiators for use in photopolymerization are ethyl-4-dimethyl-aminobenzoate and diethyl-aminoethyl methacrylate. In some embodiments, the ratio of photoiniator to amine is about 2:1, about 1:1, or about 1:2.

In some embodiments, the free radical polymerization may be initiated by reaction of an amine with peroxide. In some embodiments, the monomer comprises one or more functional groups selected from, without limitation, urethane, amine, acrylic, carboxylic, amide and hydroxyl. In some embodiments, the monomer is present in the composition in an amount of between about 1% to about 40%, by total weight, or, in some embodiments, between about 10% to about 30%, by weight, or, in some embodiments, between about 12% to about 20%, by total weight.

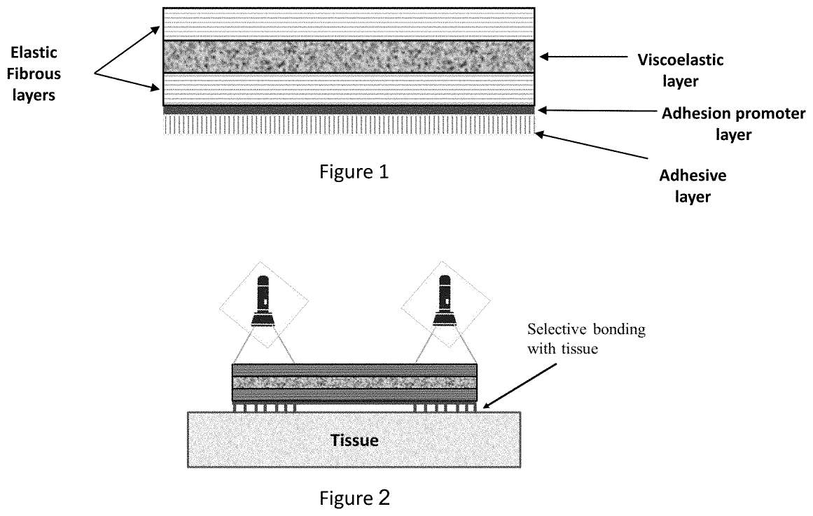

In some embodiments, the adhesive comprises a cross-linker. In some embodiments, cross-linker allows the polymerize function of an adhesive. In some embodiments, the cross-linker comprises functional groups which can cross-link one or more of the monomers, or oligomer (e.g., thermoplastic resin). In some embodiments, the adhesion promoter allows to selectively bond the disclosed matrix to selected areas of the biological tissue by activation, (e.g., upon applying light irradiation), as demonstrated e.g., in FIG. 1.

In some embodiments, by “selectively”, or any grammatical derivative thereof, it is meant to refer to being adhesive in selected portion(s) of the matrix, thereby allowing a desired portion of the matrix to adhere or to affix to e.g., a damaged tissue so as to prevent leakage and/or to allow regeneration of a new tissue.

As used herein, the term “polymeric strand” refers to any composition of monomeric units covalently bound to define a backbone.

In some embodiments, the cross-linker comprises functional groups selected from, but are not limited to, hydroxyl and acrylic. In some embodiments, the cross linker is selected from, but is not limited to, multifunctional acrylates, preferably tri- or tetrafunctional acrylates. In some embodiments, the cross linker is present in the composition in an amount of between about 0.1% to 5.0%, by total weight or, in some embodiments from 0.5% and 2.0%, by total weight, including any value and range therebetween.

In some embodiments, the composition-of-matter comprises an adhesion promoter.

In some embodiments, the composition-of-matter comprises an adhesive and an adhesion promoter. In some embodiments, the composition-of-matter comprises an adhesive and is devoid of adhesion promoter.

In some embodiments, the adhesion promoter is incorporated in a portion of the adhesive. In some embodiments, the adhesion promoter is incorporated in a portion of the adhesive, in an amount of e.g., 0.01% to 10%, or 01% to 3%, or 0.1% to 5%, by weight. In some embodiments, the adhesion promoter is attached on a surface of the adhesive.

The term “adhesion promoter”, as used herein, is known in the art and refers to a reagent that can catalyze or form a bond. The adhesion promoter may be useful for the preparation of high quality adhesive with improved processability.

In some embodiments, the adhesion promoter is attached on a surface of the adhesive. In some embodiments, the adhesion promoter attached on a surface of the adhesive is in the form of a solution. Non-limiting exemplary solution comprises silane or a derivative thereof (e.g., 1% to 5%, by weight), alcohol (e.g., about 15%, by weight) and water (e.g., about 1%, by weight). In some embodiments, the solution is acidic (e.g., having pH of about 4). Further embodiments of this aspect of present disclosure are included hereinbelow, under “the preparation process” and in the Examples section that follows, and form an integral part of embodiments relating to attaching the promoter onto a surface of the adhesive.

In some embodiments, the adhesion promoter incorporated in the adhesive is or comprises silane or a derivative thereof. In some embodiments, the silane derivative is selected from, without being limited thereto, vinyltriethoxysilane, triethoxysilylpropyl, or 3-aminopropyltriethoxysilane, which may allow e.g., a condensation reaction with a polar group, e.g., amine group or epoxy.

In some embodiments, the adhesion promoter comprises an aminosilane and one or more of epoxy, vinyl, sulfur, methacryl, acetoxy, isocyanurate or polyethyleneoxide functionality.

In some embodiments, and without being bound by any particular mechanism, the adhesion promoter (e.g., vinyltriethoxysilane) can undergo condensation and also additional polymerization (e.g., by vinyl group which has a double coupling). In some embodiments, the adhesion promoter may comprise more than one type of silane derivative.

In some embodiments, the disclosed composition-of-matter further comprises a filler. The term “filler” describes an inert material modifies the properties of a polymeric material and/or adjusts a quality of the end products. The filler may be an inorganic particle, for example calcium carbonate, silica and clay.

Fillers may be added to the modeling formulation in order to reduce shrinkage during polymerization or during cooling, for example, to reduce the coefficient of thermal expansion, increase strength, increase thermal stability, reduce cost and/or adopt rheological properties.

In some embodiments, the filler is selected from, without being limited thereto, quartz, silica-glass comprising e.g., strontium, barium, zinc, boron and yttrium, aluminoborosilicate glass, and/or colloidal silica. In some embodiments, the filler is in the form of one or more particles. In some embodiments, the one or more particles features a size of at least one dimension thereof (e.g., diameter, length) that ranges from about 10 nm to 100 μm, or, in some embodiments, from about 30 nm to about 30 μm, e.g., 30 nm, 40 nm, 50 nm, 60 nm, 70 nm, 80 nm, 100 nm, 200 nm, 300 nm, 400 nm, 500 nm, 600 nm, 700 nm, 800 nm, 900 nm, 1 μm, 5 μm, 10 μm, 15 μm, 20 μm, 25 μm, or 30 μm, including any value and range therebetween.

In some embodiments, the filler particles are coated with a coupling agent to bond to the resin matrix, e.g., a silyl group (sometimes referred to as “silanized” filler, as is known in the art). In some embodiments, the filler particles are uncoated.

In some embodiments, the adhesive includes, but is not limited to, thermoplastic resin.

Typical suitable thermoplastic resins useful for implementing an adhesive layer of the present invention include but are not limited to alkyds such as those derived from esterification of polybasic acids, as for example, phthalic anhydride, fumaric acid, maleic anhydride, isophthalic acid, terephthalic acid, trimesic acid, hemimellitic acid, succinic anyhydride, fatty acids derived from mineral or vegetable oils and the like, and polyhydric alcohols as for example glycerol, ethylene glycol, propylene glycol, pinacol, 1,4-butanediol, 1,3-propanediol, sorbitol, pentaerythritol, 1,2-cyclohexanediol and the like.

Still other typical suitable thermoplastic resins useful for implementing an adhesive layer include, but are not limited to, unsaturated polyesters derived from reaction of dibasic acids such as maleic anhydride, fumaric acid, adipic acid, azelaic acid and the like, and dihydric alcohols such as ethylene glycol and propylene glycol, 1,3-butylene glycol, 2,3-butylene glycol, diethylene glycol, dipropylene glycols and the like; and silicones such as dimethyldichlorosilane and the like.

Further typical suitable thermoplastic resins useful for implementing an adhesive layer of the present invention include, but are not limited to, polylactones such as poly(pivalolactone), poly(ε-caprolactone) and the like; polyurethanes derived from reaction of diisocyanates such as 1,5-naphalene diisocyanate, p-phenylene diisocyanate, m-phenylene diisocyanate, 2,4-toluene diisocyanate, 4,4′ diphenylmethane diisocyanate, 3-3′-dimethyl-4,4′-diphenyl-methane diisocyanate, 3,3′dimethyl-4,4′biphenyl diisocyanate, 4,4′ diphenylisopropylidiene diisocyanate, 3,3′-dimethyl-4,4′-diphenyl diisocyanate, 3,3′-dimethyl-4,4′-diphenylmethane diisocyanate, 3,3′-dimethoxy-4,4′-biphenyl diisocyanate, dianisidine diisocyanate, tolidine diisocyanate, hexamethylene diisocyanate, 4,4′-diisocyananodiphenylmethane and the like and linear long-chain diols such as poly(tetramethylene adipate), poly(ethylene adipate), poly(1,4-butylene adipate), poly(1,5-pentylene adipate), poly(1,3 butylene adipate), poly(ethylene succinate), poly(2,3-butylene succinate), polyether diols and the like; polycarbonates such as polymethane bis(4-phenyl)carbonate), poly(1,1-ether bis(4-phenyl)carbonate), poly(diphenylmethane bis(4-phenyl)carbonate, poly-1,1-eye lohexane bis(4-phenyl)carbonate and the like; polysulfones; polyether ether ketones; polyamides such as poly(4-amino butyric acid), poly(hexamethylene adipamide), poly(6-aminohexanoic acid), poly(m-xylylene adipamide), poly(p-xylylene sebacamide), poly(2,2,2-trimethyl hexamethylene terephthalamide), poly(metaphenyleneisophthalamide), poly(p-phenylene terephthalamide), and the like; polyesters such as poly(ethylene azelate), poly(ethylene-1,5-naphthalate), poly(1,4-cyclohexane dimethylene terephthalate), poly(ethylene oxybenzoate), poly(para-hydroxy benzoate), poly(1,4-cyclohexylidene dimethylene terephthalate), poly(1,4-cyclohexylidene dimethylene terephthalate) polyethylene terephthalate, polybutylene terephthalate and the like; poly(arylene oxides) such as poly(2,6-dimethyl-1,4-phenylene oxide), poly(2,6-diphenyl-1,4-phenylene oxide) and the like; poly(arylene sulfides) such as poly(phenylene sulfide) and the like; polyetherimides; thermoplastic elastomers such as polyurethane elastomers, fluoroelastomers, butadiene/acrylonitrile elastomers, silicone elastomers, polybutadiene, polyisobutylene, ethylene-propylene copolymers, ethylene-propylene-diene polymers, polychloroprene, polysulflde elastomers, block copolymers, made up of segments of glassy or crystalline blocks such as polystyrene, poly(vinyl-toluene), poly(t-butyl styrene), polyester and the like and the elastomeric blocks such as polybutadiene, polyisoprene, ethylene-propylene copolymers, ethylene-butylene copolymers, polyether ester and the like as for example the copolymers in polystyrene-polybutadiene-polystyrene block copolymer manufactured by Shell Chemical Company under the trade name of Kraton®; vinyl polymers and their copolymers such as polyvinyl acetate, polyvinyl alcohol, polyvinyl chloride, polyvinyl butyral, polyvinyl-idene chloride, ethylene-vinyl acetate copolymers, and the like; polyacrylics, polyacrylate and their copolymers such as polyethyl acrylate, poly(n-butyl acrylate), polymethyl methacrylate, polyethyl methacrylate, poly(n-butyl methacrylate), poly(n-propyl methacrylate), polyacryl-amide, polyacrylonitrile, polyacrylic acid, ethylene-acrylic acid copolymers, methyl methacrylate-styrene copolymers, ethylene-ethyl acrylate copolymers, methacrylated budadiene-styrene copolymers and the like; polyolefins such as low density polyethylene, polypropylene, chlorinated low density polyethylene, poly(4-methyl-1-pentene) and the like; ionomers; and polyepichlorohydrins; polycarbonates and the like.

In some embodiments, the thermoplastic resin is selected from, without being limited thereto, the group consisting of bisphenol-A-dimethacrylate, bisphenylglycidyl methacrylate, mono- and multi-functional aliphatic and aromatic urethane acrylate oligomers, epoxy-acrylate oligomers and urethano-acrylate oligomers. In some embodiments, units of which the thermoplastic resin is composed is characterized by an average molecular weight (MW) of between about 100 and about 5000, or in some embodiments, between about 500 and about 3000.

Herein, the term “average molecular weight” is meant to refer to weight average molecular weight in Daltons (Da). As used herein, the term “weight average molecular weight”, in some embodiments, refers to a molecular weight measurement that depends on the contributions of polymer molecules according to their sizes.

In some embodiments, the thermoplastic resin comprises substantially only one type of oligomer or polymer. In some embodiments, the thermoplastic resin comprises a mixture of different polymers or oligomers.

By “different type”, herein it is meant that the polymers have a different chemical composition, or, in some embodiments, different in structural factors such as, and without limitation, molecular weight or end groups.

In some embodiments, the thermoplastic resin comprises one or more functional groups selected from, without being limited thereto, urethane, amine, acrylic, amide, and hydroxyl. In some embodiments, the thermoplastic resin is present in the composition in an amount of between about 1% to about 30%, by total weight, or about 5% to about 25%, by total weight, or, in some embodiments, about 10% to about 18%, by total weight.

Preparation Process:

Any of the fibers described herein (according to any one of the respective embodiments) may optionally be produced by any suitable technique for preparing fibers (including macro-sized fibers, micro-sized fibers and nano-sized fibers), such as conventional fiber-spinning techniques. Such techniques include, for example, solution spinning, electrospinning, wet spinning, dry spinning, melt spinning and gel spinning. Each spinning method imparts specific physical dimensions and mechanical properties of the resulting fibers, and can be tuned to give the desired characteristics according to the required application of the fibers and layer of fibers described herein.

Briefly, a fiber spinning technique optionally involves the use of spinnerets. These are similar, in principle, to a bathroom shower head, and may have from one to several hundred small holes. As the filaments, or crude fibers, emerge from the holes in the spinneret, the dissolved or liquefied polymer is converted first to a rubbery state and then solidified. This process of extrusion and solidification of “endless” crude fibers is called spinning, not to be confused with the textile operation of the same name, where short pieces of staple fiber are twisted into yarn.

Wet spinning is used for fiber-forming substances that have been dissolved in a solvent. The spinnerets are submerged in a chemical bath and as the filaments emerge they precipitate from solution and solidify. Because the solution is extruded directly into the precipitating liquid, this process for making fibers is called wet spinning. Fibers such as, for example, acrylic, rayon, aramid, modacrylic and spandex can be produced by this process.

Dry spinning is also used for fiber-forming substances in solution, however, instead of precipitating the polymer by dilution or chemical reaction, solidification is achieved by evaporating the solvent in a stream of air or inert gas. The filaments do not come in contact with a precipitating liquid, eliminating the need for drying and easing solvent recovery. This process may be used for the production of, for example, acetate, triacetate, acrylic, modacrylic, PBI, spandex and vinyon.

In melt spinning, the fiber-forming substance is melted for extrusion through the spinneret and then the crude fibers directly solidified by cooling. Melt spun crude fibers can be extruded from the spinneret in different cross-sectional shapes (round, trilobal, pentagonal, octagonal and others). Nylon (polyamide), olefin, polyester, saran and sulfar, for example, are produced in this manner. Non-polymeric fibers can also be produced by melt-spinning.

Gel spinning is a special process used to obtain high strength or other special fiber properties. The polymer is not in a true liquid state during extrusion. Not completely separated, as they would be in a true solution, the polymer chains are bound together at various points in liquid crystal form. This produces strong inter-chain forces in the resulting filaments that can significantly increase the tensile strength of the fibers. In addition, the liquid crystals are aligned along the fiber axis by the shear forces during extrusion. The filaments emerge with an unusually high degree of orientation relative to each other which increases their strength. The process can also be described as dry-wet spinning, since the filaments first pass through air and then are cooled further in a liquid bath. Some high-strength polyethylene and aramid fibers, for example, are produced by gel spinning.

Alternatively, the fibers can be of natural or synthetic origins, and can be provided ready for use without further manipulation or preparation procedures or upon surface treatment thereof.

In some embodiments of any one of the embodiments described herein, the fibers are formed of electrospun polymeric material.

As used herein, the terms “electrospin”, “electrospinning”, “electrospun” and the like refer to a technology which produces fibers (e.g., nanofibers) from a polymer solution. During this process, one or more polymers of the polymeric material as described herein are liquefied (i.e., melted or dissolved) and placed in a dispenser. An electrostatic field is employed to generate a positively charged jet from the dispenser to the collector. Thus, a dispenser (e.g., a syringe with metallic needle) is typically connected to a source of high voltage, preferably of positive polarity, while the collector is grounded, thus forming an electrostatic field between the dispenser and the collector. Alternatively, the dispenser can be grounded while the collector is connected to a source of high voltage, preferably with negative polarity. As will be appreciated by one ordinarily skilled in the art, any of the above configurations establishes motion of positively charged jet from the dispenser to the collector. Reverse polarity for establishing motions of a negatively charged jet from the dispenser to the collector is also contemplated. At the critical voltage, the charge repulsion begins to overcome the surface tension of the liquid drop. The charged jets depart from the dispenser and travel within the electrostatic field towards the collector. Moving with high velocity in the inter-electrode space, the jet stretches and the solvent therein evaporates, thus forming fibers which are collected on the collector, e.g., in a form of a layer of fibers.

Several parameters may affect the diameter of the fiber, these include, the size of the dispensing hole of the dispenser, the dispensing rate, the strength of the electrostatic field, the distance between the dispenser and/or the concentration of the polymeric material used for fabricating the electrospun fiber.

The dispenser can be, for example, a syringe with a metal needle or a bath provided with one or more capillary apertures from which the liquefied polymeric material as described herein can be extruded, e.g., under the action of hydrostatic pressure, mechanical pressure, air pressure and high voltage.

According to one embodiment, the collector is a rotating collector which serves for collecting the electrospun fibers thereupon. Employing a rotating collector can result in a layer of electrospun fibers with a continuous gradient of porosity. Such a porosity gradient can be achieved by continuous variation in the velocity of the collector or by a longitudinal motion of the dispenser, these result in a substantial variation in the density and/or spatial distribution of the fibers on the collector and thus, result in a porosity gradient along the radial direction or along the longitudinal direction of the collector, respectively. Typically, but not obligatorily, the rotating collector has a cylindrical shape (e.g., a drum); however, it will be appreciated that the rotating collector can be also of a planar geometry.

According to another embodiment, the collector is a flat ground collector which serves for collecting the electrospun scaffold thereupon. Employing a flat ground collector enables collection of random nanofibers. It will be appreciated that the flat ground collector is typically a horizontal collector or a vertical collector.

In some embodiments of any one of the embodiments described herein, any two or more adjacent layers formed of fibers (including elastic layers and/or viscoelastic layers according to any of the respective embodiments described herein) are optionally prepared by continuous electrospinning.

It is to be appreciated that a viscoelastic layer formed of fibers does not necessarily retain a fibrous structure. For example, as exemplified herein, a viscoelastic layer in a form of a continuous film may be formed from fibers which then merge, thereby losing some or all of the porous and fibrous nature of the layer.

According to an aspect of some embodiments of the invention, there is provided a process of preparing a composition-of-matter and/or core matrix according to any of the respective embodiments described herein, the process comprising forming the one or more elastic layers (e.g., made of polymeric fibers, according to any of the respective embodiments described herein) and the viscoelastic layer(s) by continuous electrospinning, thereby forming the composition-of-matter and/or core matrix.

According to some embodiments of the invention, there is provided a process of preparing a composition-of-matter and/or core matrix according to any of the respective embodiments described herein, the process providing the one or more elastic layers and the viscoelastic layer(s) (according to any of the respective embodiments described herein), placing the viscoelastic layer(s) parallel to the elastic layers (optionally between the elastic layers), e.g., in a stacked formation, and pressing the elastic layers and the viscoelastic layer(s) together, thereby forming the composition-of-matter and/or core matrix. In some such embodiments, the process further comprises forming the elastic layers by electrospinning.

In some embodiments, pressing the elastic layers and viscoelastic layer(s) together comprises applying a pressure of at least 1 gram/cm². In some embodiments, the pressure is at least 2 gram/cm². In some embodiments, the pressure is at least 4 gram/cm². In some embodiments, the pressure is at least 8 gram/cm².

In some embodiments, the process further comprises heating the viscoelastic layer prior to, concomitantly with, and/or subsequently to pressing the layers. In some such embodiments, the heating is to a temperature which is above a glass transition temperature and/or melting point (optionally a glass transition temperature) of a polymer in the viscoelastic layer, in accordance with any of the respective embodiments described herein (e.g., 40° C.).

In some embodiments, the process further comprises depositing an adhesion promoter, and/or an adhesive on a layer of the elastic polymeric material. In some embodiments, the process further comprises applying a radiation (e.g., U.V. radiation) to activate the adhesive.

In exemplary embodiments, the elastic layer is treated with silane, thereby allowing to increase the adhesion property of a surface of the elastic layer.

Optional Applications:

According to another aspect of embodiments of the invention there is provided an article-of-manufacture comprising a composition-of-matter and/or matrix according to any of the respective embodiments described herein.

In some such embodiments, the article-of-manufacture consists essentially of the composition-of-matter, as described herein. In some such embodiments, the article-of-manufacture comprises additional components in addition the composition-of-matter, as described herein.

Examples of articles-of-manufacture in which a flexible composition-of-matter according to any of the respective embodiments described herein may be advantageously incorporated include, without limitation, articles intended to be applied to surfaces of various shapes, such as packaging materials, coatings, adhesive tape, sealants; articles comprising an inflatable component, such as a balloon (e.g., balloon catheters); and devices with movable parts (wherein the composition-of-matter may optionally be attached to two or more separately movable parts), such as household and/or industrial machinery.

In some embodiments of any one of the embodiments described herein, the article-of-manufacture is a medical device. In some such embodiments, the medical device is an implantable medical device.

In some embodiments of any one of embodiments described herein relating to a medical device, the medical device is for use in the field of general surgery, neurology, ear-nose and throat, urology, gynecology/obstetrics, thoracic, dental/maxillofacial, gastroenterology, plastic surgery, ophthalmology, cardiovascular and/or orthopedic medicine.

In some embodiments of any one of the embodiments described herein, the article-of-manufacture (e.g., medical device) is identified for use in a treatment. In some embodiments, the article-of-manufacture (e.g., medical device) is identified for use in repairing and/or substituting a biological tissue.

According to another aspect of embodiments of the invention, there is provided a method of repairing and/or substituting a biological tissue in a subject in need thereof, the method comprising contacting the biological tissue with article-of-manufacture (e.g., medical device) described herein.

In some embodiments, the method comprises affixing at least a portion of article-of-manufacture in/on a biological tissue. In some embodiments, the affixing is performed by curing. In some embodiments, the affixing is performed by light (e.g., UV) radiation on the portion comprising the adhesion promoter.

In some embodiments of any of the embodiments described herein relating to repairing and/or substituting a biological tissue, a biological tissue to be repaired and/or substituted is a membrane (e.g., following traumatic injury, hernia and/or surgical incision of the membrane). In some embodiments, the membrane to be repaired and/or substituted is dura mater (e.g., following traumatic injury and/or surgical incision of the dura mater). In some embodiments, the article-of-manufacture has a sheet-like geometry (e.g., as described herein) which mimics that of a membrane.

Examples of treatments for which an article-of-manufacture according to such embodiments may be used (e.g., by implantation and/or temporary internal or topical use) in a treatment or method described herein (according to any of the respective embodiments) include, without limitation, repairing and/or substituting a biological tissue, such as dural repair, hernia repair, internal and/or topical wound closure, skin closure and/or repair (e.g., as part of plastic surgery), supporting another medical implant (such as in breast reconstruction surgery), sealing tissues and/or organs in order to contain bodily fluids and/or air (e.g., treating bile duct leakage), sealing an anastomosis, inhibition of post-surgical adhesions between tissues and promotion of hemostasis (e.g., wherein the matrix is coated with thrombin and/or fibrinogen and/or fibrin); as well as administration of a therapeutically effective agent (e.g., by incorporating the therapeutically effective agent in and/or on the core matrix, according to any of the embodiments described herein relating to inclusion of an additional ingredient).

Examples of treatments for which an implantable medical device according to embodiments described herein may be identified for use include, without limitation, dural repair, hernia repair, internal wound closure, sealing tissues and/or organs in order to contain bodily fluids and/or air, sealing an anastomosis, inhibition of post-surgical adhesions between tissues, promotion of hemostasis, and administration of a therapeutically effective agent.

In some embodiments, the medical device is configured for eluting a therapeutically active agent, e.g., an agent included as an additional ingredient according to any of the respective embodiments described herein. In some such embodiments, the medical device is a stent. Optionally, the composition-of-matter forms at least a portion of a flexible sleeve of the stent.

The therapeutically active agent may optionally be incorporated within a core matrix and/or on a surface of the core matrix. Optionally, the therapeutically active agent is incorporated within a drug-eluting layer within the core matrix and/or on a surface of the core matrix. Such a drug eluting layer may be formed of any suitable substance known in the art of drug-eluting layers.

Herein, the phrase “repairing and/or substituting a biological tissue” refers to repair of tissue which is physically damaged in any manner, and encompasses supporting and/or holding damaged tissue together in vivo or ex vivo, as well as filling gaps formed by an absence of tissue (substituting tissue). The damaged tissue may be damaged, for example, by detachment (e.g., tearing, cutting), compressive stress, tensile stress, shear stress, cellular dysfunction and/or cell death.

In some embodiments of any of the embodiments described herein relating to repairing and/or substituting a biological tissue, the repairing and/or substituting a biological tissue comprises suturing the article-of-manufacture to the tissue (that is, the article-of-manufacture and tissue are attached via at least one suture).

As exemplified herein, core matrices described herein are particularly suitable for being sutured without losing mechanical or functional integrity.

As used herein the term “about” refers to ±10%.

The terms “comprises”, “comprising”, “includes”, “including”, “having” and their conjugates mean “including but not limited to”. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a polymer” or “at least one polymer” may include a plurality of polymers, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

Materials:

Dimethylformamide was obtained from Sigma Aldrich (Israel). Dioxane was obtained from Sigma Aldrich (Israel). Tetrahydrofuran was obtained from Sigma Aldrich (Israel). Poly(ε-caprolactone-co-L-lactic acid-co-glycolic acid-co-trimethylene carbonate) was obtained from/by Poly-Med Inc. (USA). Poly(L-lactic acid) was obtained from NatureWorks (USA). Poly(DL-lactic acid-co-ε-caprolactone) was obtained from Lactel (USA). Poly(L-lactic acid-co-ε-caprolactone) was obtained from Purac Biomaterials (Netherlands).

Determination of Structure and Morphology:

Samples of individual sheets or 3-layer patches were coated with gold and characterized using a Quanta 200 environmental scanning electron microscope (SEM) with a tungsten filament (FEI). Fiber size and mean pore size were measured using ImageJ software.

Mechanical Properties:

Tensile tests (strain ramp) were carried out using a custom-made uniaxial tensile machine (equipped with a 25 kg load cell) in accordance with ASTM international standard D882-12 for testing tensile properties of thin plastic sheeting. Patches were cut in a dog bone configuration and thickness was measured at three points along the neck of the dog bone. The samples were immersed in PBS (phosphate buffer saline) at a temperature of 37° C. for 15 minutes before the test, and then mounted on the clamps of the machine. Each sample was stretched until breakage. The sample's Young's modulus (elastic modulus), ultimate tensile strength (UTS) and elongation at failure were determined.

Shear loss (G′) and shear storage (G″) modulus of the middle layer were evaluated via shear rheometer. The measurements were conducted using a strain-controlled rotational rheometer (AR-G2, TA Instruments), with a stainless-steel parallel plate geometry (20 mm), which includes a Peltier temperature control. All tests were conducted at a temperature of 10° C. A strain sweep and frequency sweep tests were conducted to determine the linear viscoelastic regime of the layer. Time and temperature sweep tests were then performed at a strain range of 0.1-0.7% and a frequency of 0.05-1 Hz.

Suture Retention Test:

Suture retention tests were based on the method described in the ANSI/AAMI/ISO 7198:1998/2001/(R) 2004 standard. Samples were cut and conditioned as described hereinabove for the uniaxial tensile test. One end of the dog bone shaped sample was removed by scalpel and the sample was sutured (Premilene® 4/0 suture) at a minimum distance of 2 mm from its free end. The sample was then placed on the tensile machine, by connecting the patch to the first grip and the suture to the other grip. A tensile test was then conducted as described hereinabove, in order to measure the force at failure of the samples.

Statistical Methods:

All final values describe the average of a minimum of 3 test items. Results are expressed as the mean values±standard error.

### Example 1

Electrospun elastic sheets were prepared as previously described (WO 2015/092797). The following polymers were electrospun using the previously described general procedure (molecular weights herein refer to weight average molecular weights, except when indicated otherwise):

PLLA—poly(L-lactic acid) homopolymer (molecular weight 150±5 kDa);

PLLA/CL—poly(L-lactic acid-co-ε-caprolactone) (molar ratio 70:30 lactic acid:caprolactone, molecular weight 210±10 kDa);

PCL/LLA/GA/TMC—poly(ε-caprolactone-co-L-lactic acid-co-glycolic acid-co-trimethylene carbonate) linear block copolymer (molar ratio 35:34:17:14 caprolactone:lactic acid:glycolic acid:trimethylene carbonate, molecular weight 165±5 kDa, number average molecular weight 90±5 kDa);

Next, viscoelastic sheets prepared by film casting or electrospinning as previously WO 2015/092797.

### Example 2

### Activating the Adhesive Layer

Nanofiber patches were tested for lap shear tests after radiation to activate adhesive layer. The patches were silane treated to increase the adhesion to the adhesive.

Materials:

Nanofiber patches; High Q Bond—Permanent Adhesive Resin Cement (Dual cure)—BJM Lab; 3-methacryl oxypropyl-trimethoxysilane (MEMO); N-2-Aminoethyl-3-aminopropyltrimethoxysilane (DAMO).

Methodology:

Silane solutions were prepared using 1 and 2% of DAMO and MEMO silanes in a mixture of 99% ethanol and 1% of water. The patches were immersed in the silane solutions and dried at room temperature (RT) for 24 hours, and contact angles were measured.

FIGS. 1 and 2 present schemes of the integral 3-layerd matrix with an adhesive layer that can be activated selectively with radiation curing in selected areas.

Double lap shear samples were prepared using strips of PC as substrate, as can be seen in FIG. 2. Samples were exposed to UV lamp for 2 minutes in order to cure the adhesive. The samples were tensile tested to determine lap shear properties.

Results:

The results of silane treatment are presented in Table 1 below. Best results (lower contact angle) were obtained for 2% MEMO.

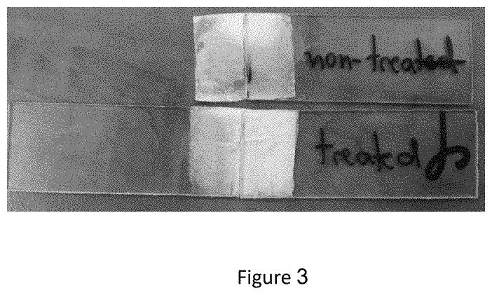

Results of lap-shear tests are shown in Table 2 below. It can be seen that 2% silane treated patch enabled an increase of 6.5 folds in the adhesion strength compared to non-treated samples. FIG. 3 depicts samples of treated and non-treated patches after the tests. It can be clearly seen the adhesive failure of the non-treated patch.

TABLE 1
Contact angle results of silane treated patches

| Sample | Concentration (%) | Contact angle |
|---|---|---|
| Reference | — | 103 |
| Silane DAMO | 1% | 103 |
| | 2% | 97 |
| Silane MEMO | 1% | 87 |
| | 2% | 86 |

TABLE 2
Lap-shear tests results

| Sample | Stress at max. load (MPa) |
|---|---|
| Reference patch | 0.013 |
| Silane treated patch | 0.084 |

### Example 3

### Adhesion Tests

Nanofiber patches were tested for adhesion to a fascia tissue in a lap shear test. The patches were silane treated to increase the adhesion to the adhesive. Patches silane treated and non-treated were compared. Samples without adhesive were also tested for comparison.

Materials:

Nanofiber patches; High Q Bond—Permanent Adhesive Resin Cement (Dual cure)—BJM Lab; 3-methacryl oxypropyl-trimethoxysilane (MEMO); Bovine Fascia tissue.

Methodology:

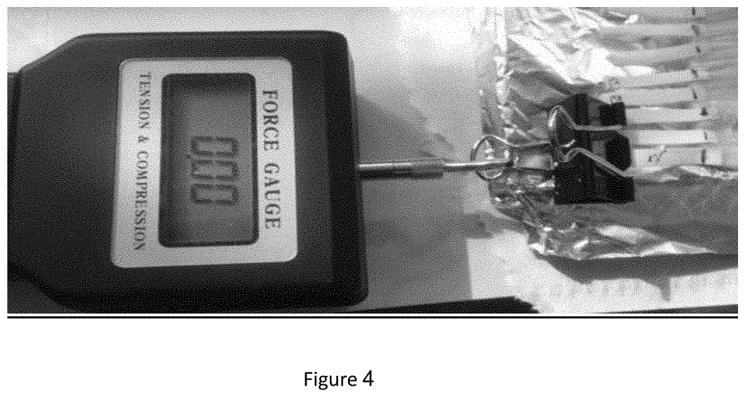

Silane solution was prepared using 2% of MEMO in a mixture of 99% ethanol and 1% of water. The patches were immersed in the silane solution and dried at RT for 24 hours. Lap shear samples were prepared using strips of nanofiber patches, as shown in FIG. 4. Strips from nanofiber patches (silane treated and non-treated) were cut (7 mm L×3 mm W) and an area of 4 mm L×3 mm W was bonded to fascia tissue. Samples were exposed to UV lamp for 5 minutes in order to cure the adhesive. The samples were thereafter tensile tested to determine lap shear properties using a Force Gauge Lutron FG-5000A.

Results:

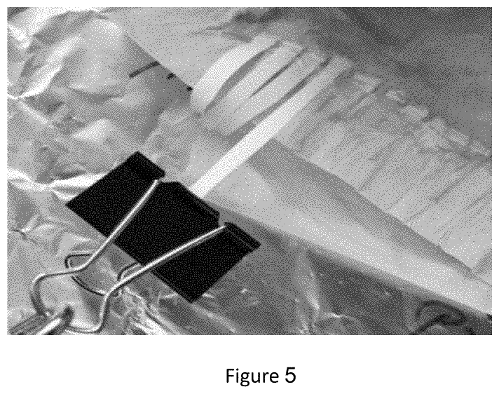

The results of the shear tests can be seen in Table 3 below. A weak adhesion was obtained for the samples without adhesive. A higher adhesion strength could be noticed for the samples bonded to the fascia with the adhesive. The patches failed before debonding, shows a substantial elongation during the tests, as shown in FIG. 5. The best results were obtained for silane treated patches.

TABLE 3
Lap-shear tests results (average of 5 samples)

| Sample | Max. load (N) | Shear strength (MPa) |
|---|---|---|
| Non-treated patch without adhesive | 0.94 | 0.078 |
| Non-treated patch with adhesive | 1.54 | 0.128 |
| Silane treated patch with adhesive | 2.46 | 0.205 |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A composition-of-matter comprising a multi-layer matrix, said matrix comprising:
(i) at least two layers of an elastic polymeric material independently made of polymeric fibers,
(ii) at least one layer of a viscoelastic polymeric material interposed between two of the layers of the elastic polymeric material, and
(iii) at least one adhesive promoter and/or adhesive layer, applied on, or within, at least one surface of at least one of the elastic polymeric material.

2. The composition-of-matter of claim 1, wherein said adhesive layer is selected from (i) an adhesive layer comprising a mixture or a blend of an elastic polymeric material and an adhesive material; (ii) an adhesive layer comprising one or more materials selected from: thrombin, fibrinogen, an albumin, gelatin, a polysaccharide or a derivative thereof, hydroxysuccinimide (NHS) ester, poly(vinyl acetate), poly(vinyl pyrrolidone, a polyester, polyethyleneglycol, polyaminoacid, poly(glutamic acid), poly(L-aspartic acid), multi-arms PEG, or any combination thereof; (iii) an adhesive layer comprising one or more growth factors.

3. (canceled)

4. (canceled)

5. The composition-of-matter of claim 1, wherein said layer of said viscoelastic polymeric material is selected from: (i) a viscoelastic polymeric material comprising a polymer characterized by a glass transition temperature and/or melting point at a temperature below 40° C.; (ii) a viscoelastic polymeric material characterized by a loss tangent (G″/G′) at a temperature of 10° C. and frequency of 0.1 Hz which is in a range of from 0.01 to 4; (iii) a viscoelastic polymeric material characterized by at least one of: a) a storage shear modulus (G′) in a range of from 0.01 to 10 MPa, at a temperature of 10° C. and frequency of 0.1 Hz; and b) a loss shear modulus (G″) in a range of from 0.0001 to 2 MPa, at a temperature of 10° C. and frequency of 0.1 Hz; (iv) a viscoelastic polymeric material comprising poly(lactic acid-co-ε-caprolactone).

6. The composition-of-matter of claim 1, wherein said elastic polymeric material is selected from: (i) elastic polymeric material comprising a polymer characterized by a glass transition temperature and/or melting point at a temperature above 40° C.; (ii) an elastic polymeric material being a porous layer characterized by a porosity of at least 50%; and (iii) an elastic polymeric material selected from the group consisting of: poly(lactic acid-co-ε-caprolactone), poly(ε-caprolactone-co-L-lactic acid-co-glycolic acid-co-trimethylene carbonate), mixtures of poly(lactic acid-co-ε-caprolactone) and poly(lactic acid), and mixtures of poly(ε-caprolactone-co-L-lactic acid-co-glycolic acid-co-trimethylene carbonate) and poly(lactic acid), or any mixture thereof.

7. (canceled)

8. (canceled)

9. The composition-of-matter of claim 1, wherein said polymeric fibers are characterized by a mean diameter in a range of from 0.001 to 30 μm.

10. (canceled)

11. (canceled)

12. (canceled)

13. The composition-of-matter of claim 1, wherein each of said layers of an elastic polymeric material is selected from any one of: (i) an elastic polymeric material characterized by a thickness in a range of from 10 to 500 μm; (ii) an elastic polymeric material characterized by an elastic modulus in a range of from 1 kPa to 10 MPa; (iii) an elastic polymeric material characterized by an elongation at failure of at least 100%; (iv) an elastic polymeric material characterized by an ultimate tensile strength of at least 0.05 MPa; (v) a recovery of at least 75.

14. (canceled)

15. (canceled)

16. (canceled)

17. (canceled)

18. (canceled)

19. (canceled)

20. The composition-of-matter of claim 1, wherein said matrix is characterized by a water-permeability of less than 1 ml per hour per cm² upon exposure to an aqueous liquid at a pressure of 40 mmHg.

21. The composition-of-matter of claim 1, wherein said adhesive layer is or comprises a polymerizable resin selected from the group consisting of a polymerizable monomer and a filler.

22. The composition-of-matter of claim 1, wherein said at least one adhesion promoter comprises a vinyl group.

23. The composition-of-matter of claim 1, wherein said at least one adhesion promoter is or comprises a silane compound or a derivative thereof.

24. The composition-of-matter of claim 23, wherein said derivative of said silane comprises one or more functional groups selected from the group consisting of amine, epoxy, or polyolefin.

25. The composition-of-matter of claim 23, wherein said derivative of said silane is selected from the group consisting of vinyltriethoxysilane, triethoxysilylpropyl, and 3-aminopropyltriethoxysilane.

26. The composition-of-matter of claim 21, wherein the polymerization of said polymerizable resin is affected by irradiation.

27. The composition-of-matter of claim 26, wherein said irradiation is selected from thermal irradiation and light irradiation.

28. (canceled)

29. The composition-of-matter of claim 21, wherein said polymerizable monomer is selected from the group consisting of methyl methacrylate, triethylene glycol dimethacrylate, 2-hydroxyethyl methacrylate, hexanediol methacrylate, dodecanediol dimethacrylate, bisphenol-A-dimethacrylate, bisphenylglycidyl methacrylate, mono- and urethane acrylate, urethane dimethacrylate, epoxy-acrylate and urethano-acrylate.

30. An article-of-manufacture comprising the composition-of-matter of claim 1.

31. (canceled)

32. (canceled)

33. A method of repairing and/or substituting a biological tissue in a subject in need thereof, the method comprising contacting the biological tissue with the article-of-manufacture of claim 30, and affixing at least a portion of article-of-manufacture in/on a biological tissue thereby repairing and/or substituting the biological tissue.

34. The method of claim 33, wherein said affixing is performed by curing.

35. The method of claim 34, wherein said curing is thermal curing.

36. The method of claim 34, wherein said affixing is performed by light radiation of said adhesion promoter.
